(19) United States
(12) Patent Application Publication
(10) Pub. No.: US 2012/0329770 A1
(43) Pub. Date: Dec. 27, 2012

(54) CEPHALOSPORIN DERIVATIVES USEFUL AS BETA-LACTAMASE INHIBITORS AND COMPOSITIONS AND METHODS OF USE THEREOF

(76) Inventors: Gary Igor Dmitrienko, Waterloo (CA); Ahmad Ghavami, Guelph (CA); Valerie Joy Goodfellow, Waterloo (CA); Jarrod W. Johnson, Port Colborne (CA); Anthony Paul Krismanich, Waterloo (CA); Laura Marrone, Waterloo (CA); Thammaiah Viswanatha, Waterloo (CA); Sundaramma Viswanatha, Waterloo (CA)

(21) Appl. No.: 13/581,301

(22) PCT Filed: Feb. 25, 2011
(86) PCT No.: PCT/CA11/50115

§ 371 Date: Aug. 24, 2012

Related U.S. Application Data
(60) Provisional application No. 61/282,539, filed on Feb. 26, 2010.

(51) Int. Cl. A61K 31/545; A61K 31/546; A61P 31/04; C07D 501/60

(52) U.S. Cl. 514/206; 540/230; 514/209; 540/228; 514/207; 540/227; 540/229; 514/208

(57) ABSTRACT

The present invention relates to cephalosporin derivatives having β-lactamase inhibitory activity. The compounds are useful in preventing or treating bacterial resistance to an antibiotic, e.g. a β-lactam antibiotic. Disclosed herein are compounds that are inhibitors of class B metallo-β-lactamases, as well as class A, C, and D serine β-lactamases. In some preferred embodiments, the compounds are 3′-thiobenzoate derivatives of a cephalosporin. Pharmaceutical compositions, methods, uses, kits and commercial packages comprising the compounds are also disclosed.

### CROSS REFERENCE TO RELATED APPLICATIONS

This application claims the benefit of priority of U.S. Provisional Patent Application No. 61/282,539 filed Feb. 26, 2010, which is incorporated herein by reference in its entirety.

### FIELD

The present disclosure relates to cephalosporin derivatives having β-lactamase inhibitory activity. The compounds are useful for inhibiting β-lactamase in vitro and/or in vivo and, in particular, for preventing or treating bacterial resistance to an antibiotic (e.g. a β-lactam antibiotic).

### BACKGROUND

The β-lactam antibiotics constitute one of the three largest classes of clinically useful antibiotics along with the fluoroquinolones and macrolides. It is estimated that >50% of all antibiotic prescriptions are for β-lactams. Since the discovery of the naturally occurring penicillins such as penicillin G, a number of significant structural variants, each retaining the essential β-lactam ring, have been discovered and have found specific niches in chemotherapeutic applications (FIG. 1). Dalhoff et al. provide a recent overview of the development of the major classes of β-lactam antibiotics from a medicinal chemistry perspective (Dalhoff, A.; Thomson, C. J. Chemotherapy 2003, 49, 105-120).

Since their introduction into standard clinical practice, shortly after the second world war, these antibiotics, which combine the remarkable properties of oral bioavailability (in most cases), high antibiotic potency and relatively low toxicity to the host, have had an enormous impact on the maintenance of human health. As a result, the prospect that bacteria can develop or acquire high levels of resistance to these and other antibiotics is indeed disquieting. For reviews of resistance to β-lactam antibiotics see: (Walsh), T. R. Int. J. Antimicrob. Agents 2010, 36, Suppl. 3 S8-14. (b) Bush, K. Clin. Microbial. Infect. 2008, 14 (Suppl. 1), 134-143. (c) Fisher, J. F.; Meroueh, S. O.; Mobashery, S. Chem. Rev. 2005, 105, 395-424 and references to earlier reviews therein. (d) Poole, K. Cell Mol. Life Sci. 2004, 61, 2200-2223. (e) Hancock, R. Trends Microbial. 1997, 5, 37-42. A brief but interesting history of the discovery of the major classes of clinically useful antibiotics and the emergence of resistance to them is presented by Walsh and Wright in the preface to the February 2005 issue of Chemical Reviews which is devoted entirely to reviews of antibiotic resistance mechanisms (Walsh, C. T.; Wright, G. D. Chem. Rev. (Editorial) 2005, 105, 391-394).

Various studies have revealed that antibiotic resistance arises typically by three mechanisms: 1) active trans-membrane efflux of the drug; 2) reduction in sensitivity to the drug by modification of the antibiotic target through mutation; and 3) expression of enzymes capable of destruction of the antibiotic ((a) Fisher, J. F.; Meroueh, S. O.; Mobashery, S. Chem. Rev. 2005, 105, 395-424 and references to earlier reviews therein; (b) Poole, K. Cell Mol Life Sci. 2004, 61, 2200-2223; (c) Hancock, R. Trends Microbial. 1997, 5, 37-42). In the case of the β-lactam antibiotics, it has been shown that all three mechanisms play a role to varying degrees. It is generally agreed that the third mechanism, mediated in this case by a variety of hydrolytic enzymes collectively referred to as the β-lactamases, is the single most important cause of high level bacterial resistance to β-lactams.

The ability of some bacteria to effect inactivation of β-lactam antibiotics, through hydrolysis of the β-lactam ring system in penicillins to yield the corresponding penicilloic acid (FIG. 2), was noted very early on in the history of the study of these microbial natural products (Abraham, E. P.; Chain, E. B. Nature 1940, 146, 837).

Since those very early indications of the existence of such a potential resistance mechanism, widespread use and abuse of these antibiotics has led to the emergence of a large number of bacterial strains exhibiting high levels of resistance to β-lactams as a consequence of harbouring a β-lactamase gene. It has been estimated that the number of known β-lactamases is approaching 900 (www.lahey.org/studies). The recognition that some β-lactamase genes are plasmid-encoded raised concerns in the early 1980s that horizontal transfer of the antibiotic-resistance genes would lead to proliferation of β-lactam antibiotic resistant organisms. This has indeed proven to be the case, and from the mid-1980s to 2000, the number of different plasmid-mediated β-lactamases detected in clinical isolates rose from 19 to 255 (Payne, D. J.; Du, W.; Bateson, J. H. Exp. Opin. Invest. Drugs, 2000, 9, 247-261).

The β-lactamases are divided into four classes based on sequence homology (Ambler, R. P. Philos. Trans. R. Soc. London, Ser. B, 1980, 289, 321-331). The class A, C and D classes are all enzymes that employ an active site serine residue as a nucleophile in their catalytic mechanism, in a process somewhat akin to the well-known chymotrypsin “acyl enzyme” mechanism. The class B enzymes employ an active site zinc ion in their catalytic apparatus (FIG. 2). The β-lactamases which were first recognized as therapeutic problems were largely of the A type, so initial efforts at combating β-lactam antibiotic resistance were focused on the serine enzymes.

A number of lines of investigation led to the discovery of several so-called mechanism-based inhibitors for the serine β-lactamases, such as sulbactam, tazobactam and clavulanic acid (FIG. 3). These, used in combination with existing penicillins, have served remarkably well to allay the concerns about (β-lactamase resistance for the past 25 years, since their introduction into clinical use. For the most part, the class A β-lactamases have remained susceptible to these inhibitors, although a number of reports of inhibitor-resistant class A (IRT-type)-producing organisms have appeared (Arpin, C.; Labia, R.; Dubois, V.; Noury, P.; Souquet, M.; Quentin, C. Antimicrob. Agents Chemother. 2002, 46, 1183-1189).

In parallel with the development of (β-lactamase inhibitors, extensive efforts in various pharmaceutical laboratories to modify the β-lactam systems in order to create antibiotics with broader antibiotic spectrum and lower susceptibility to the (β-lactamases were carried out with significant success. Of particular interest was the development of the carbapenems (e.g. imipenem and meropenem and doripenem, FIG. 1), which have emerged as “drugs of last resort” in treatment of serious infections by antibiotic resistant organisms (Edwards, J. R.; Betts, M. J. J. Antimcrob. Chemother. 2000, 45, 1-4; Oelschlaeger, P.; Ai, N.; DuPrez, K.; Welsh, W. J.; Toney, J. H. J. Med. Chem. 2010, 53, 3013-3027).

The common occurrence of β-lactamase-producing organisms in hospital settings has led to the significant use of the carbapenems to treat serious hospital-acquired, known as nosocomial, infections. Among the serious nosocomial infections are those caused by opportunistic bacteria which are normally harmless towards healthy individuals but which cause serious, potentially fatal, infection in patients with diminished immune systems, including burn victims, AIDS patients, cancer patients, transplant patients and those with lung diseases such as cystic fibrosis.

The emergence of bacteria capable of producing β-lactamases with potent carbapenemase activity has created a great deal of concern about the possibility of the development of high level resistance to these drugs of last resort, leaving the antibiotic cupboard essentially bare in the event of serious nosocomial infections ((a) Queenan, A. M.; Bush, K. Clin. Microbiol. Rev. 2007, 28, 440-458. (b) Jones R. N.; Biedenbach, D. J.; Sader, H. S.; Fritsche, T. R.; Toleman, M. A.; Walsh, T. R. Diagn. Microbiol Infect Dis. 2005, 51, 77-84. (c) Livermore, D. M.; Woodford, N. Curr. Opin. Microbiol. 2000, 5, 489-495. (d) Livermore, D. M. Clin. Infect. Dis. 2002, 34, 634-640. (e) Nordmann, P.; Poirel, L. Clin. Microbiol. Infect. 2002, 8, 321-331).

The emergence of serine-β-lactamases (SBLs) and metallo-β-lactamases (MBLs) with a broad spectrum of activity encompassing all known classes of β-lactam antibiotics requires renewed efforts to establish strategies with potential for overcoming this growing threat through development of broad spectrum β-lactamase inhibitors which are effective against both the serine and the metallo-β-lactamases.

In particular, there is an urgent need for β-lactamase inhibitors which are effective against the metallo-β-lactamases that, without exception, are potent carbapenem-hydrolyzing enzymes and that are encoded on transferable plasmids. Of the ten MBLs currently identified on transferable plasmids (e.g. IMP, VIM, GIM, SPM, SIM, KHM, AIM, DIM, TMB, and NDM types), four currently (e.g. IMP, VIM, SPM and NDM enzymes) represent the most immediate threat (Walsh, T. R. Int. J. Antimicrob. Agents, 2010, 36, Uppl. 3, S8-14). Although the IMP and VIM enzymes have been identified in certain isolates of Enterobacteriacae, their clinical significance is primarily due to their presence in multi-resistant strains of non-fermenting Gram-negative bacteria (Pseudomonas and Acinetobacter spp.) that are notoriously difficult to treat with antibiotics. The incidence of MBLs in Enterobacteriacae has, however, increased dramatically recently with the discovery of the plasmid-mediated NDM-1 enzyme in multiple strains of a variety of organisms including E. coli, K pneumonia and lesser pathogens such as Enterobacter aerogenes and Proteus mirabilis. NDM-1 confers high level resistance to all carbapenems and is associated with a multiple resistance phenotype to most commonly used antibiotic classes including fluoroquinolones and aminoglycosides. (Yong, D. et al. Antimicrob. Agents Chemother.2009, 53, 5046-5054; Kumarasamy, K. K. et al. Lancet Infect. Dis. 2010, 10, 597-602.; Sidjabat, H. et al. Clin. Infect. Dis. 2011, 52, 481-484; Nordmann, P. et al. J. Clin. Microbiol., 2011, 49, 718-721)

In light of the foregoing, there remains a need for broad-spectrum inhibitors of clinically important (β-lactamases, including the class A, class C, and class D serine β-lactamases and the class B metallo-β-lactamases (MBLs). In particular, there remains a need for effective inhibitors of the Class B metallo-β-lactamases.

### SUMMARY

The present disclosure relates to cephalosporin derivatives having β-lactamase inhibitory activity, e.g. β-lactamase inhibitors. Compounds, pharmaceutical compositions, methods, uses, kits and commercial packages are some of the aspects disclosed herein.

In a first aspect, there is provided a compound of Formula (I) for use in inhibiting a β-lactamase and/or preventing or treating bacterial resistance to an antibiotic:

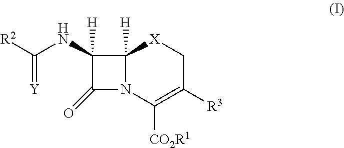

wherein
X is selected from O, S, S═O, SO₂, C═O, C═S, CR⁴R⁵, where R⁴ and are independently H or C₁—C₅ alkyl, or NR⁶ where R⁶ is where R⁶ is C(═O)R⁷ or SO₂R⁷ and R⁷ is H or lower alkyl;
Y is selected from O or S;
R¹ is selected from H and a pharmaceutically acceptable cation;
R² is selected from

—CH₂-aryl, —CH₂dihydro-aryl or —CH₂-heteroaryl,

—(CR′R″)ₙ-aryl or —(CR′R″)ₙ-heteroaryl, where n=0, 1, 2, 3 or 4 and where R′ and R″ are independently C₁—C₅ alkyl, hydroxy, alkoxy, or cyano,

—C(═N—OR^{′″})-aryl, —C(═N—OR^{′″})-heteroaryl or —C(CH₃)₂—C(═O)OR^{′″} where R′ is H, —C₁—C₅ alkyl, fluoromethyl, CH₂C(CH₃)₂CO₂H or CH₂CO₂H,

cyanomethyl, cyanomethylthiomethyl or dihalomethylthiomethyl, trihalomethylthiomethyl, or

a radical found in a cephalosporin antibiotic, for example, any one of the following

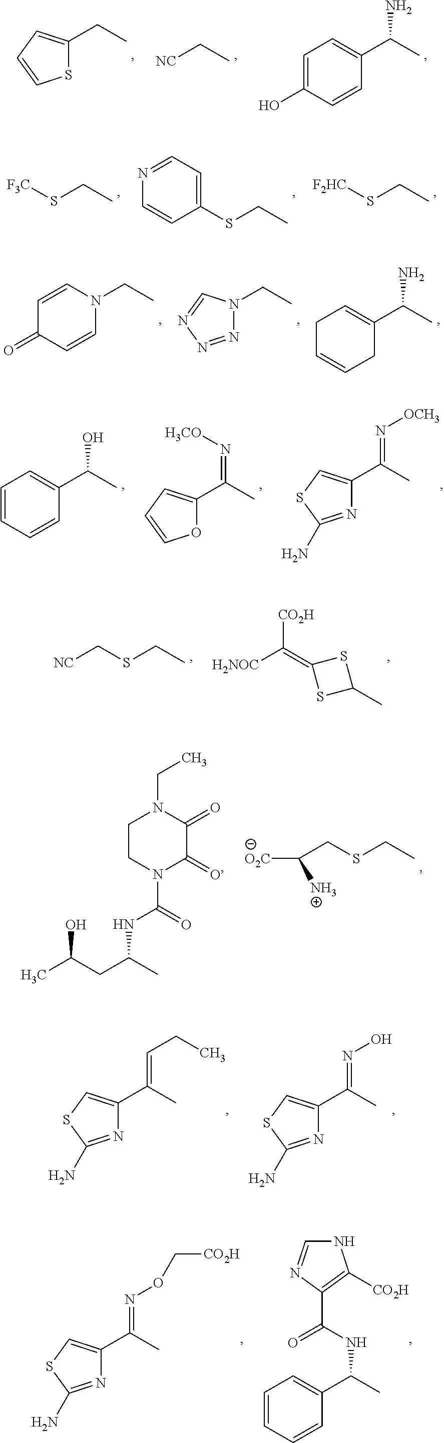

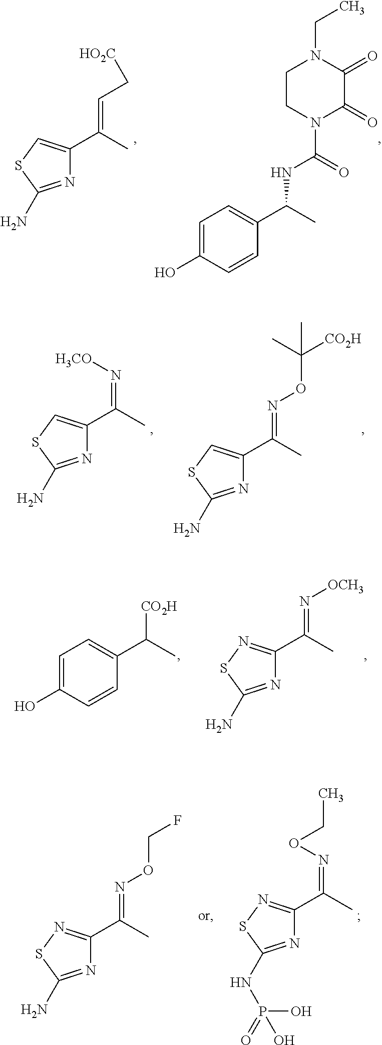

R³ is CR⁸R⁹Z, wherein R⁸ is H or —C₁—C₅ alkyl, R⁹ is H or —C₁—C₅ alkyl, and Z is selected from —S—C(═O)R¹⁰, —S—C(═S)R¹⁰, —SR¹⁰, —SOR¹⁰, —SO₂R¹⁰ or R¹⁰ where R¹⁰ is selected from aryl, preferably having 6 or more carbons, heteroaryl, preferably having 6 or more ring atoms, (CH₂)ₙ-aryl (where n=1 to 5), (CH₂)ₙ-heteroaryl (where n=1 to 5), O(CH₂)ₙH (where n=1 to 5), S(CH₂)ₙH (where n=1 to 5), O(CH₂)ₙaryl (where n=0 to 5), S(CH₂)ₙaryl (where n=0 to 5), O(CH₂)ₙheteroaryl (where n=0 to 5) or S(CH₂)ₙheteroaryl (where n=0 to 5), or —O—NHC(═O)R¹¹, —N(SO₂R¹¹)₂ or —OSO₂R¹¹ wherein R″ is selected from —C₁—C₅ alkyl, —CF₃, —CCl₃, aryl, heteroaryl, —(CH₂)ₙ-aryl (wherein n=1 to 5), —(CH₂)ₙ-heteroaryl (wherein n=1 to 5), —O(CH₂)ₙH (wherein n=1 to 5), —S(CH₂)ₙH (wherein n=1 to 5), —O(CH₂)ₙaryl (wherein n=0 to 5), —S(CH₂)ₙaryl (wherein n=0 to 5), —O(CH₂)ₙheteroaryl (wherein n=0 to 5) or —S(CH₂)ₙheteroaryl (wherein n=0 to 5),
wherein alkyl, aryl and heteroaryl may be substituted or unsubstituted;
or a pharmaceutically acceptable salt or ester thereof

In another aspect, there is provided a compound of Formula (I):

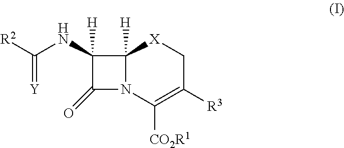

wherein
X is O or S;
Y is O or S;
R¹ is selected from H and a pharmaceutically acceptable cation;
R² is selected from:

—CH₂-aryl, —CH₂dihydro-aryl or —CH₂-heteroaryl,

—(CR′R″)ₙ-aryl or —(CR′R″)ₙ-heteroaryl, where n=0, 1, 2, 3 or 4 and where R′ and R″ are independently C₁-C₅ alkyl, hydroxy, alkoxy, or cyano,

—C(═N—OR^{′″})-aryl, —C(═N—OR^{′″})-heteroaryl or —C(CH₃)₂—C(═O)OR^{′″} where R^{′″} is H, —C₁-C₅ alkyl, fluoromethyl, CH₂C(CH₃)₂CO₂H or CH₂CO₂H,

cyanomethyl, cyanomethylthiomethyl or dihalomethylthiomethyl, trihalomethylthiomethyl, or

a radical found in a cephalosporin antibiotic, for example, any one of the following

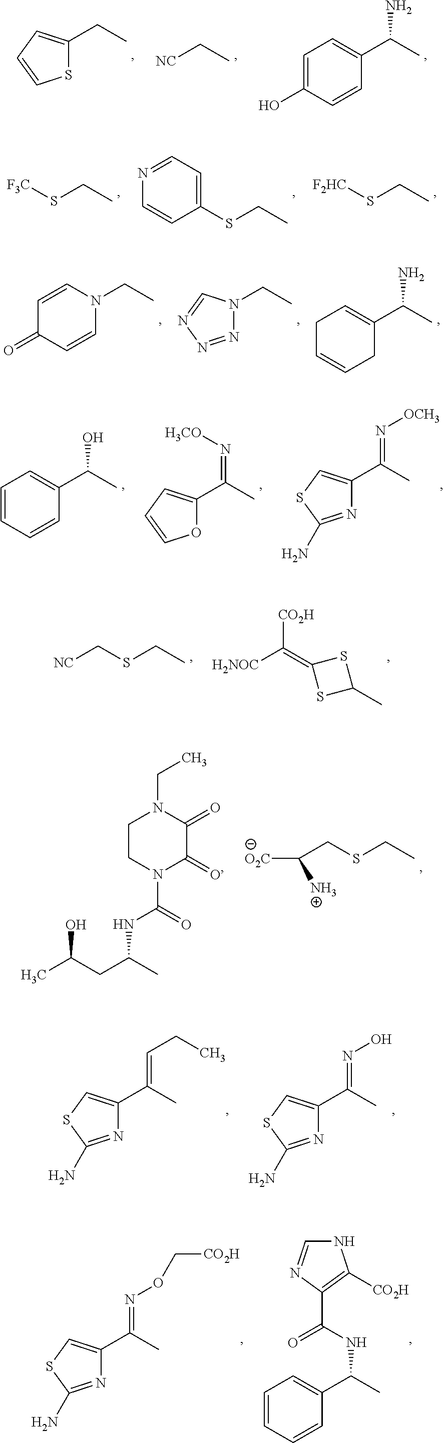

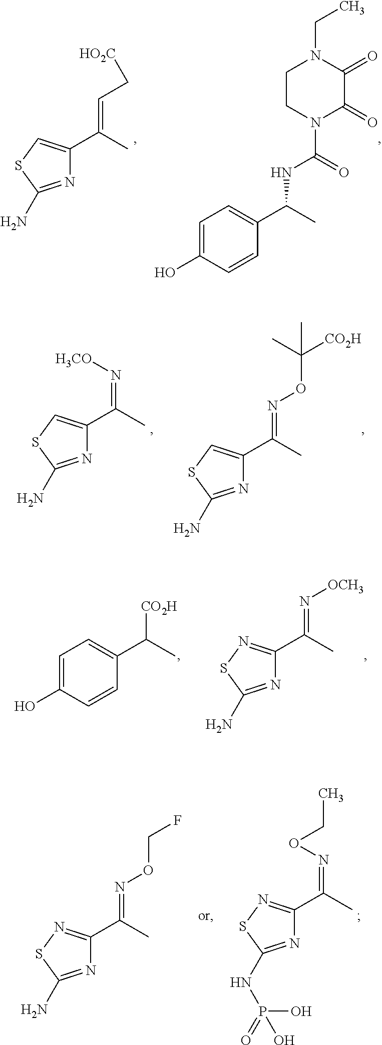

R³ is CR⁸R⁹Z, wherein R⁸ is H or —C₁-C₅ alkyl, R⁹ is H or —C₁-C₅ alkyl, and Z is selected from —S—C(═O)R¹⁰, —S—C(═S)R¹⁰, —SR¹⁰, —SOR¹⁰, —SO₂R¹⁰ or R¹⁰ where R¹⁰ is selected from aryl, preferably having 6 or more carbons, heteroaryl, preferably having 6 or more ring atoms, (CH₂)ₙ-aryl (where n=1 to 5), (CH₂)ₙ-heteroaryl (where n=1 to 5), O(CH₂)ₙH (where n=1 to 5), S(CH₂)ₙH (where n=1 to 5), O(CH₂)ₙaryl (where n=0 to 5), S(CH₂)ₙaryl (where n=0 to 5), O(CH₂)ₙheteroaryl (where n=0 to 5) or S(CH₂)ₙheteroaryl (where n=0 to 5), or —O—NHC(═O)R¹¹, —N(SO₂R¹¹)₂ or —OSO₂R¹¹ wherein R″ is selected from —C₁-C₅ alkyl, —CF₃, —CCl₃, aryl, heteroaryl, —(CH₂-aryl (wherein n=1 to 5), —(CH₂-heteroaryl (wherein n=1 to 5), —O(CH₂)ₙH (wherein n=1 to 5), —S(CH₂)ₙH (wherein n=1 to 5), —O(CH₂)ₙaryl (wherein n=0 to 5), —S(CH₂)ₙaryl (wherein n=0 to 5), —O(CH₂)ₙheteroaryl (wherein n=0 to 5) or —S(CH₂)ₙheteroaryl (wherein n=0 to 5),
wherein alkyl, aryl and heteroaryl may be substituted or unsubstituted;
or a pharmaceutically acceptable salt or ester thereof,

with the proviso that the compound is not a known cephalosporin antibiotic as disclosed herein, such as, ceftiofur, moxalactam, cefaloridin or cefalonium.

In another aspect, there is provided a compound of Formula (II)

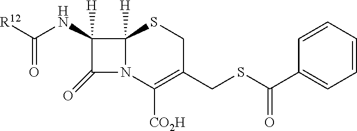

R¹² is selected from:

—CH₂-aryl, —CH₂dihydro-aryl or —CH₂-heteroaryl,

—(CR′R″)ₙ-aryl or —(CR′R″)ₙ-heteroaryl, where n=0, 1, 2, 3 or 4 and where R′ and R″ are independently C₁-C₅ alkyl, hydroxy, alkoxy, or cyano,

—C(═N—OR^{′″})-aryl, —C(═N—OR^{′″})-heteroaryl or —C(CH₃)₂—C(═O)OR^{′″} where R^{′″} is H, —C₁-C₅ alkyl, fluoromethyl, CH₂C(CH₃)₂CO₂H or CH₂CO₂H,

cyanomethyl, cyanomethylthiomethyl or dihalomethylthiomethyl, trihalomethylthiomethyl, or

a radical found in a cephalosporin antibiotic, for example, any one of the following

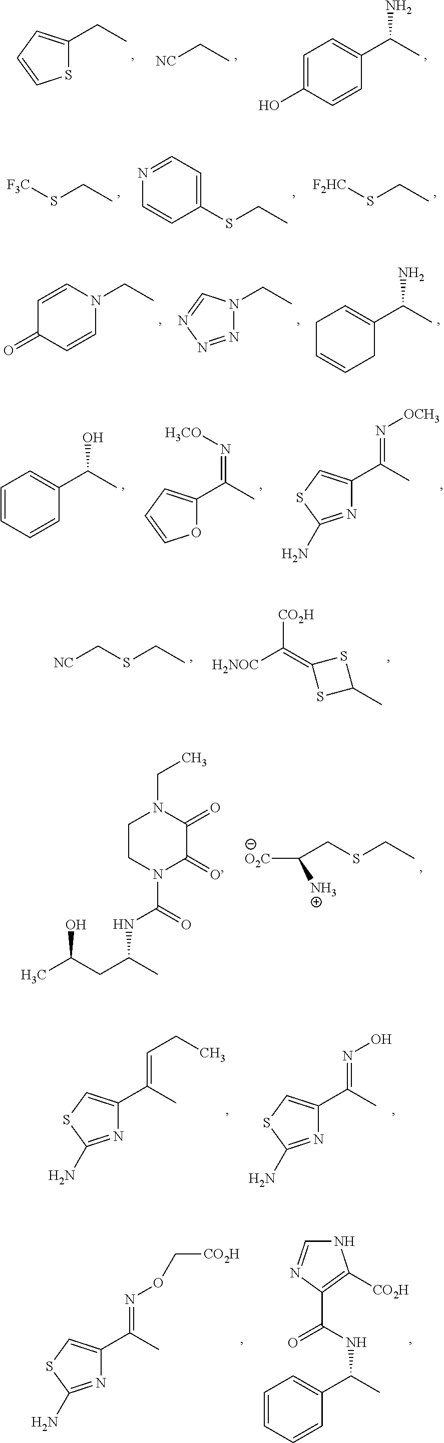

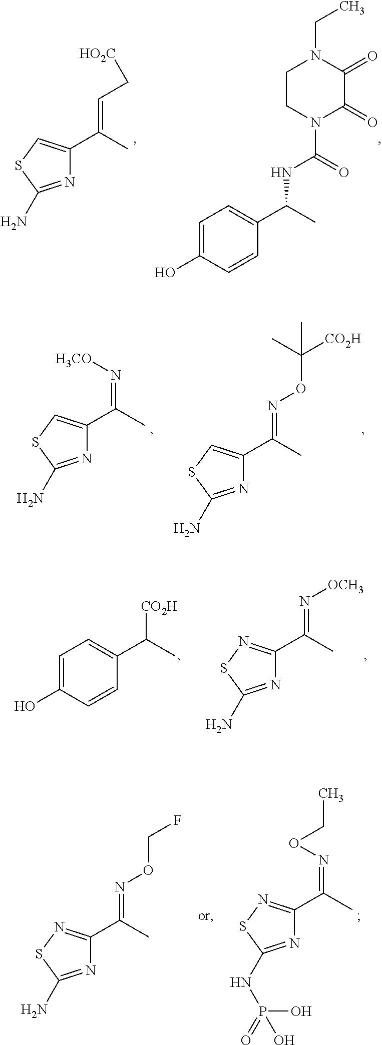

In another aspect, there is provided a compound selected from any one of:

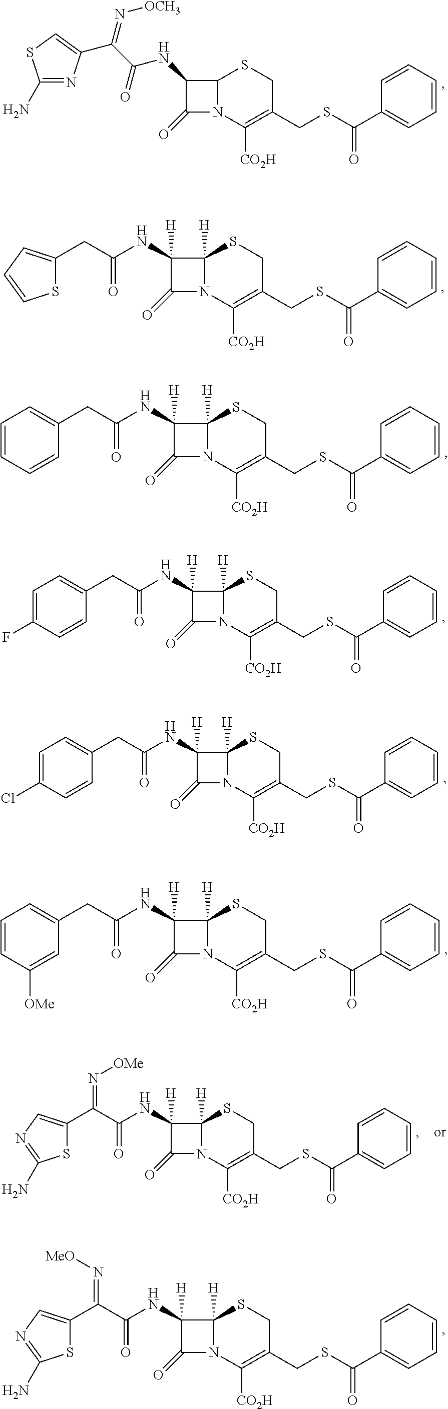

or a pharmaceutically acceptable salt or ester thereof.

In another aspect, there is provided a compound which is a C3-methybenzoylthio derivative of a cephalosporin antibiotic.

In another aspect, there is provided a pharmaceutical composition for preventing or treating bacterial resistance to an antibiotic, the composition comprising a β-lactamase inhibitory amount of a compound as defined herein, and a pharmaceutically acceptable excipient. The pharmaceutical composition may further comprise a pharmaceutically acceptable antibiotic, in particular, a β-lactam antibiotic.

In another aspect, there is provided a use of compound or a pharmaceutical composition disclosed herein in the manufacture of a medicament for treating a bacterial infection and/or preventing or treating bacterial resistance to an antibiotic.

In another aspect, there is provided a use of compound or a pharmaceutical composition disclosed herein for treating a bacterial infection and/or preventing or treating bacterial resistance to an antibiotic.

In some embodiments, the use if for treating a nosocomial bacterial infection.

In another aspect, there is provided a method of treating a bacterial infection and/or preventing or treating bacterial resistance to an antibiotic comprising administering to a patient in need thereof a β-lactamase inhibitory amount of a compound disclosed herein in combination with a therapeutically effective amount of an antibiotic.

In another aspect, there is provided a method of inhibiting a β-lactamase enzyme, the method comprising contacting the β-lactamase enzyme with a β-lactamase inhibitory amount of a compound or a pharmaceutical composition as defined herein.

In another aspect, there is provided a commercial package or kit comprising a compound or a composition as defined herein, together with instructions for use in inhibiting a β-lactamase and/or preventing or treating bacterial resistance to an antibiotic and/or treating a bacterial infection.

In another aspect, there is provided a method for synthesis of a compound disclosed herein.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 illustrates representative compounds from major structural classes of clinically useful β-lactam antibiotics.

FIG. 2 illustrates the mechanism of hydrolysis of β-lactam antibiotics by serine- and metallo-β-lactamases.

FIG. 3 illustrates some clinically useful β-lactamase inhibitors.

FIG. 4 illustrates a kinetic mechanism that characterizes the inhibition of β-lactamases by compounds of the present disclosure. β-Lactamase inhibition by a cephalosporin substrate (X) that binds tightly to the enzyme but turns over slowly. The initial E-X complex isomerizes to another more stable complex E′-X where the enzyme (now E′) is in an altered state in which the catalytic steps with X, or with the normal substrate S (e.g. meropenem), are slow. A superimposition of ab initio-optimized (RHF/6-31G(d)) structures of 3′-O-acyl (O) and 3′-S-acyl (S) cephalosporin derivatives shows the difference in conformational preference.

FIG. 5 illustrates a general strategy for the synthesis of compounds of formula (I).

FIG. 6 illustrates the differences in conformational preferences for 3′-O-acyl and 3′-S-acyl cephalosporin derivatives. A superimposition of ab initio-optimized (RHF/6-31G(d)) structures of 3′-O-acyl (O) and 3′-S-acyl (S) cephalosporin derivatives shows the difference in conformational preference.

FIG. 7 illustrates the conformations of cephalosporin derivatives in the active site of IMP-1. Shown is a superimposition of structure-optimized models of cephalosporin derivatives in the active site of IMP-1.

FIG. 8 illustrates compounds of formula that are predicted by molecular modeling studies to have enhanced affinity for β-lactamase active sites.

FIG. 9 is a scheme illustrating Inhibition of IMP-1-Catalyzed Hydrolysis of Meropenem.

FIG. 10 illustrates good protection of meropenem by a cephalosporin derivative and relatively poor protection of meropenem by ceftiofur from IMP-1-catalyzed hydrolysis in vitro. A) IMP-1 (12 nM) dependent hydrolysis of Meropenem (200 μM) in the presence/absence of Ceftiofur (100 μM) monitored at 310 nm. B) IMP-1 (12 nM) dependent hydrolysis of Meropenem (200 μM) in the presence/absence of (6R,7R)-3-(benzoylthiomethyl)-7-(2-(4-fluorophenyl)acetamido)-8-oxo-5-thia-1-azabicyclo[4.2.0]oct-2-ene-2-carboxylic acid (100 μM) monitored at 310 nm.

FIG. 11 is a scheme illustrating Inhibition of IMP-1-Catalyzed Hydrolysis of Meropenem

FIG. 12 illustrates good protection of meropenem by a cephalosporin derivative and poor protection of meropenem by moxalactam from IMP-1-catalyzed hydrolysis in vitro. A) IMP-1 (12nM) dependent hydrolysis of Meropenem (200 μM) in the presence/absence of Moxalactam (100 μM)_monitored at 310 nm. B) IMP-1 (12 nM) dependent hydrolysis of Meropenem (200 μM) in the presence/absence of (6R,7R)-3-(benzoylthiomethyl)-7-(2-(4-fluorophenyl)acetamido)-8-oxo-5-thia-1-azabicyclo[4.2.0]oct-2-ene-2-carboxylic acid (100 μM) monitored at 310 nm.

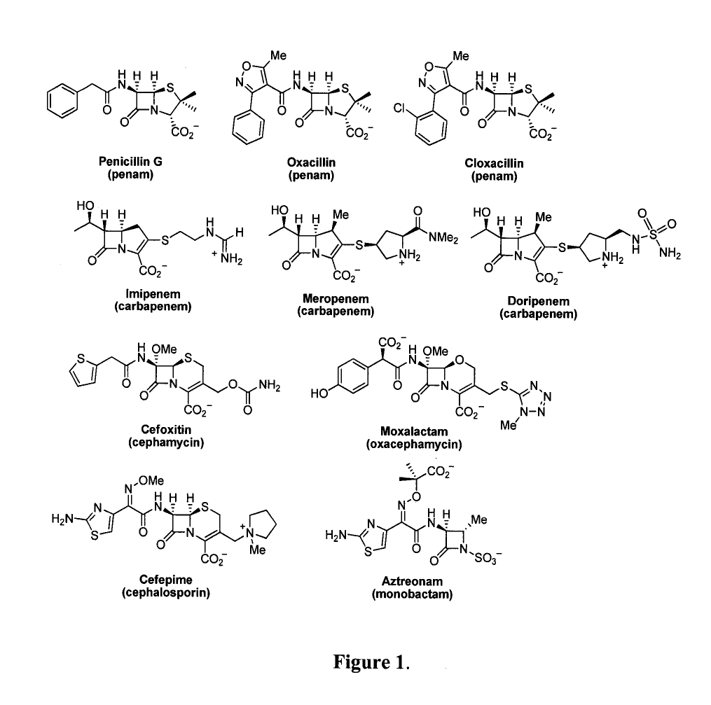

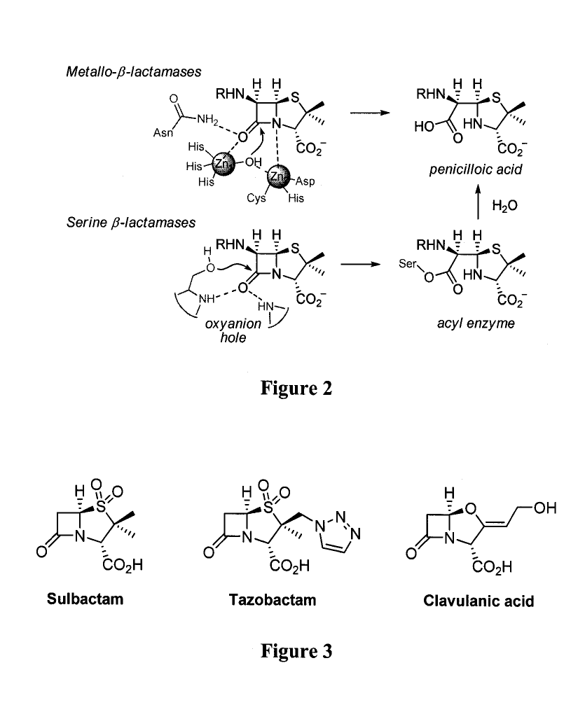

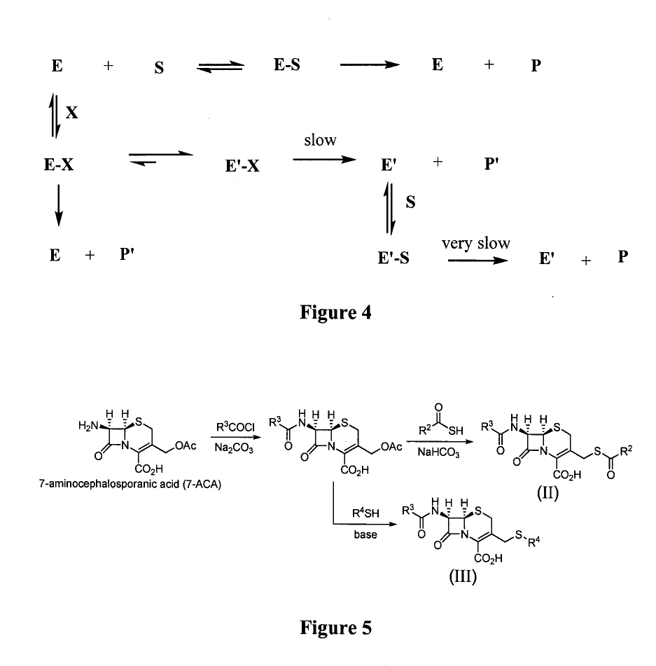

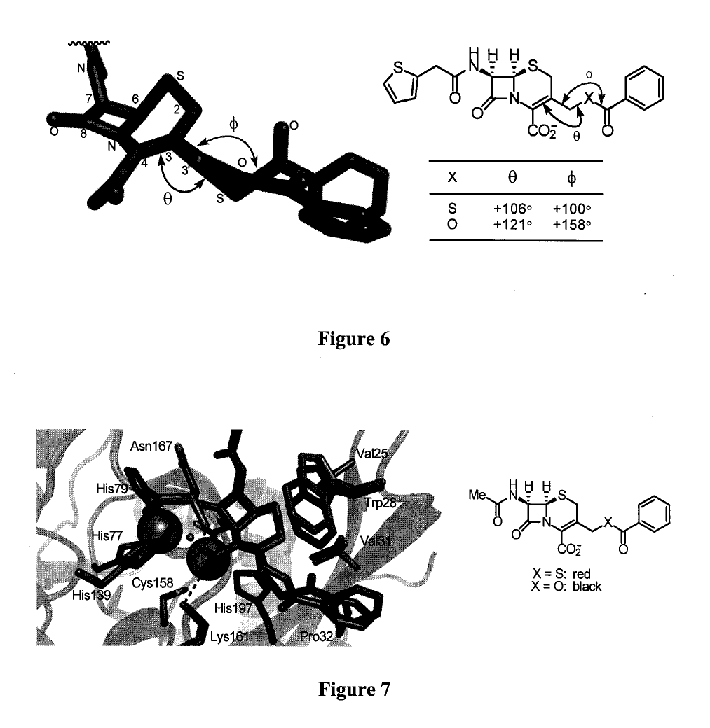

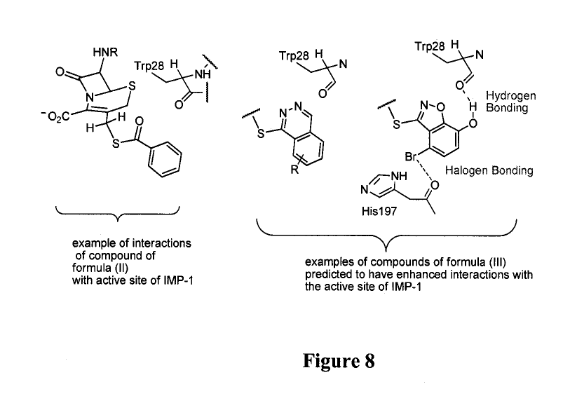

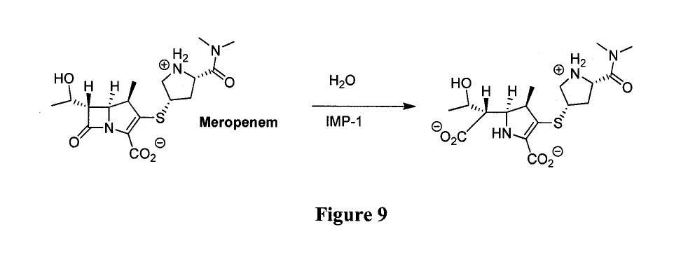

### DETAILED DESCRIPTION

Generally, the present disclosure relates to cephalosporin derivatives having β-lactamase inhibitory activity, e.g. β-lactamase inhibitors. Pharmaceutical compositions, methods, uses, kits and commercial packages comprising the inhibitors are also disclosed.

The compounds disclosed herein are useful to prevent or treat bacterial resistance to an antibiotic, in particular, bacterial resistance to β-lactam antibiotic.

The term “antibiotic” as used herein describes a compound or composition which decreases the viability of a microorganism, in particular, a bacteria, or which inhibits the growth or reproduction of a microorganism thereby increasing the generation cycle time by at least 2-fold, preferably at least 10-fold, more preferably at least 100- fold, and most preferably, kills the microorganism. An antibiotic is further in tended to include an antimicrobial, bacteriostatic, or bactericidal agent.

In preferred embodiments, the antibiotic is a β-lactam antibiotic. The term “β-lactam antibiotic” as used herein designates compounds with antibiotic properties containing a β-lactam functionality. In general, such antibiotics are prone to hydrolysis by β-lactamase enzymes, which target the β-lactam ring of the molecule. Several strains of bacteria produce β-lactamase enzymes. This contributes to “bacterial resistance” to the β-lactam antibiotic, where the antibiotic has reduced effectiveness (e.g. in inhibiting the growth or reproduction of the bacteria) due to inactivation of the antibiotic by β-lactamase.

The term “treat” as used herein means to reduce, inhibit or overcome. For example, to “treat” bacterial resistance means a reduction or complete inhibition of the rate at which an antibiotic is inactivated by a resistant microorganism, thereby resulting in prolonged or enhanced activity of the antibiotic. To “treat” a bacterial infection means a reduction or complete inhibition in the symptoms or underlying cause (e.g. bacteria) of the infection.

A “patient” may be any animal in need of treatment, such as a mammal, e.g. a dog, cat, goat, pig, horse, cow, rabbit, mouse, rat, or the like. In some embodiments, the patient is a human.

The compounds disclosed herein are cephalosporin derivatives. The expression “cephalosporin derivative” does not refer to the method of preparing the inhibitor compound (i.e. a cephalosporin antibiotic is not necessarily a starting material or an intermediate in the method of preparation) but rather indicates that the compounds share a similar core structure with cephalosporin antibiotics. However, in many embodiments, the inhibitor compound is a weak antibiotic or substantially lacks intrinsic antibiotic activity.

Thus, the compounds of the present disclosure have the general Formula (I):

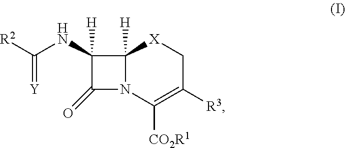

where X, Y, R¹, R² and R³ are as defined further below.

Compounds of Formula (I) disclosed herein may contain asymmetric carbon atoms. Accordingly, included herein are stereoisomeric forms of the compounds of Formula (I), including individual enantiomers and mixtures thereof, i.e. optical isomers and mixtures thereof, having β-lactamase inhibitory activity.

Unless otherwise indicated, chemical terms are used herein in accordance with their common meaning in the chemical arts.

Unless otherwise specified, the following chemical terms may encompass substituted moieties (e.g. radicals) and unsubstituted moieties, e.g. optionally substituted moieties. The terms “optional” or “optionally” means that the subsequently described event may but need not occur.

The term “alkyl” as used herein means a monovalent linear or branched hydrocarbon moiety, e.g. having from 1 to 12 carbon atoms (C₁₋₁₂ alkyl). The term alkyl includes lower alkyl, for example, moieties having from 1 to 5 carbon atoms (C₁₋₅ alkyl), from 1 to 4 carbon atoms (C₁₋₄ alkyl), from 1 to 3 carbon atoms (C₁₋₃ alkyl), from 1 to 2 carbon atoms (C₁₋₂ alkyl), or one carbon atom (C₁ alkyl), including, for example, and without being limited thereto, methyl, ethyl, propyl, iso-propyl, butyl, n-butyl, iso-butyl, sec-butyl, tert-butyl, n-pentyl, and the like. In some embodiments, the alkyl group has 1, 2, 3, 4 or 5 carbon atoms. As noted above, “alkyl” may encompass unsubstituted or substituted alkyl. Substituted alkyl may include substitution at one or more positions, e.g. 1, 2, 3, 4 positions depending on the alkyl, with a suitable substituent.

The term “halo” or “halogen”, as used herein, means a halogen radical and includes, for example, fluoro (F), chloro (Cl), bromo (Br) or iodo (I).

The term “aryl” as used herein means a carbocyclic aromatic ring system containing one or more rings wherein such rings may be attached together in a pendent manner or may be fused. For example, aryl may have 1, 2 or 3 rings. In particular embodiments, aryl is 1 ring. Aryl may have, for example, 6-18 ring atoms, e.g. 6, 10, 14 or 18 in total. The term “aryl” encompasses aromatic moieties such as phenyl, naphthyl, tetrahydronaphthyl, indanyl, biphenyl, phenanthryl, anthryl, acenaphthyl and the like. As noted above, “aryl” may encompass unsubstituted or substituted aryl. Substituted aryl may include substitution at one or more positions depending on the aryl, e.g. 1, 2, 3, or 4 positions, with a suitable substituent.

The term “substituted phenyl” means a phenyl group having one or more suitable substituents, e.g. 1, 2 or 3 substituents.

The terms ortho, meta and para apply to 1,2-, 1,3- and 1,4-disubstituted phenyl, respectively.

The term “heteroaryl”, as used herein means an aromatic ring system having at least one heteroatom, e.g. 1, 2, 3, or 4, selected from N, O and S. Heteroaryl may contain one or more rings, e.g. 1, 2 or 3 rings, wherein such rings may be attached together in a pendent manner or may be fused. The heteroaryl may have 5 to 18 ring atoms, for example, a 5 to 8 membered monocyclic or 8 to 11 membered bicyclic ring. The term “heteroaryl” encompasses heteroaromatic radicals such as pyridyl, pyridinyl, pyridonyl, pyrolyl, pyridazinyl, pyrimidinyl, pyrazinyl, pyrazolyl, imidazolyl, thiazolyl, thiophenyl, triazolyl, indolyl, indolinyl, indolonyl, indolinonyl furyl, benzofuryl, thienyl, benzothienyl, benzimidazolyl, benzothiazolyl, benzoxazolyl, benzofuranyl, benzothiophenyl, benzopyrazolyl, dihydrobenzofuranyl, dihydrobenzothiophenyl, benzooxazolonyl, quinolyl, quinolinyl, dihydroquinolinyl, tetrahydroquinoyl, isoquinolinyl, tetrahydroisoquinoyl, oxazolyl, thiazolyl, thiophenyl, benz[1,4]oxazin-3-onyl, benzodioxolyl, benz[1,3]dioxol-2-onyl, tetrahydrobenzopyranyl and phthalimidyl, and the like. The “heteroaryl” may have one or more suitable substituents, e.g. 1, 2, 3 or 4 substituents.

The term “C3-benzoylthiomethyl” (or 3′-benzoylthio, or C3-benzoylthiomethyl) refers to a sulfur-containing substituent having the formula —CH₂SC(═O)phenyl, at the C3 position on the β-lactam ring structure. The phenyl ring may be substituted or unsubstituted.

When referring to substitution, for example, of alkyl, aryl, heteroaryl or phenyl, a suitable “substituent” is a radical that does not significantly impair he function of the compound (e.g. does not significantly hinder it's ability to bind to, interact with and/or inhibit a β-lactamase enzyme). Substitution may occur independently at one or more positions, e.g. 1, 2, 3 or 4 positions depending on the moiety being substituted, with a radical such as, for example, alkyl, such as lower alkyl, carboxy, carboalkoxy, carboxamido, acyl, aryl, heteroaryl, halo, haloalkyl, haloalkoxy, hydroxy, alkyl, heteroalkyl, aryl, heteroaryl, alkoxy, thioalkoxy, amino, alkylamino, amido, cyano, nitro, oxo, carbonyl, alkoxycarbonyl, thiocarbonyl, acyl, formyl, sulfonyl, mercapto, alkylthio, alkyloxy, alkylamino, cycloalkyl, heterocycloalkyl, aryl, heteroaryl, carboxy, amino, alkylamino, dialkylamino, carbamoyl, aryloxy, heteroaryloxy, arylthio, or heteroarylthio, or the like. It will be understood by those skilled in the art that substituents can themselves be substituted, if appropriate.

It will be understood that “substitution” or “substituted with includes the implicit proviso that such substitution is in accordance with permitted valence of the substituted atom and the substituent, and that the substitution results in a stable compound.

As used herein, the definition of each expression, e.g. alkyl, n, etc., when it occurs more than once in any structure, is intended to be independent of its definition elsewhere in the same structure.

The term “pharmaceutically acceptable” is well-known in the art and generally means compatible with the other ingredients of a subject composition and not injurious to the patient.

The term “pharmaceutically acceptable salt” as used herein means a pharmaceutically acceptable salt of a compound disclosed herein. Salts may be prepared from pharmaceutically acceptable non-toxic acids and bases including inorganic and organic acids and bases. Such acids include, for example, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like fumaric, hydrochloric, hydrobromic, phosphoric, succinic, sulfuric and methanesulfonic acids. Acceptable base salts include alkali metal (e.g. sodium, potassium), alkaline earth metal (e.g. calcium, magnesium) and aluminium salts. See, e.g. P. Heinrich Stahl and Camille G. Wermuth, Handbook of Pharmaceutical Salts: Properties, Selection and Use. International Union of Pure and Applied Chemistry, Wiley—VCH 2002, and L. D. Bighley, S. M. Berge, D. C. Monkhouse, in “Encyclopedia of Pharmaceutical Technology’. Eds. J. Swarbrick and J. C. Boylan, Vol. 13, Marcel Dekker, Inc., New York, Basel, Hong Kong 1995, pp. 453-499.

A “pharmaceutically acceptable cation” refers to a pharmaceutically acceptable inorganic or organic cation. Various pharmaceutically-acceptable cations are well-known in the art. Examples of pharmaceutically acceptable monovalent inorganic cations include, but are not limited to, alkali metal ions, such as Na and K. Examples of pharmaceutically acceptable divalent inorganic cations include, but are not limited to, alkaline earth cations, such as Ca⁺² and Mg⁺². Examples of pharmaceutically acceptable organic cations include, but are not limited to, ammonium ion (i.e. NH4) and substituted ammonium ions (e.g. NH3R, NH2R2, NHR3, NR4). An example of a common quaternary ammonium ion is N(CH3)4.

Provided herein are compounds of Formula (I) which are useful in inhibiting a β-lactamase and/or preventing or treating bacterial resistance to an antibiotic:

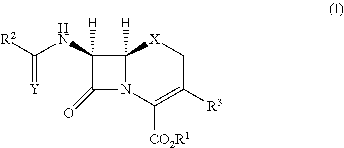

X may be selected from O, S, S═O, SO₂, C═O, C═S, CR⁴R⁵, where R⁴ and R⁵ are independently H or C₁-C₅ alkyl, or NR⁶ where R⁶ is where R⁶ is C(═O)R⁷or SO₂R⁷ and R⁷ is H or lower alkyl. In some embodiments, X is S or O. In some embodiments, X is O. In some embodiments, X is S.

Y may be selected from O or S. In some embodiments, Y is O. In some embodiments, Y is S.

R¹ is selected from H and a pharmaceutically acceptable cation. In some embodiments, the cation is an organic or an inorganic cation, for example, it may be an inorganic cation selected from a monovalent alkali metal ion, such as Na or K. Other useful cations are defined in the definitions above.

R² is selected from: —CH₂-aryl, —CH₂dihydro-aryl or —CH₂-heteroaryl; —(CR′R″)ₙ-aryl or —(CR′R″)ₙ-heteroaryl, where n=0, 1, 2, 3 or 4 and where R′ and R″ are independently C₁-C₅ alkyl, hydroxy, alkoxy, or cyano; —C(═N—OR^{′″})-aryl, —C(═N—OR^{′″})-heteroaryl or —C(CH₃)₂—C(═O)OR^{′″} where R^{′″} is H, —C₁-C₅ alkyl, fluoromethyl, CH₂C(CH₃)₂CO₂H or CH₂CO₂H; cyanomethyl, cyanomethylthiomethyl or dihalomethylthiomethyl, trihalomethylthiomethyl; or a radical selected from the following list of radicals from known cephalosporin antibiotics:

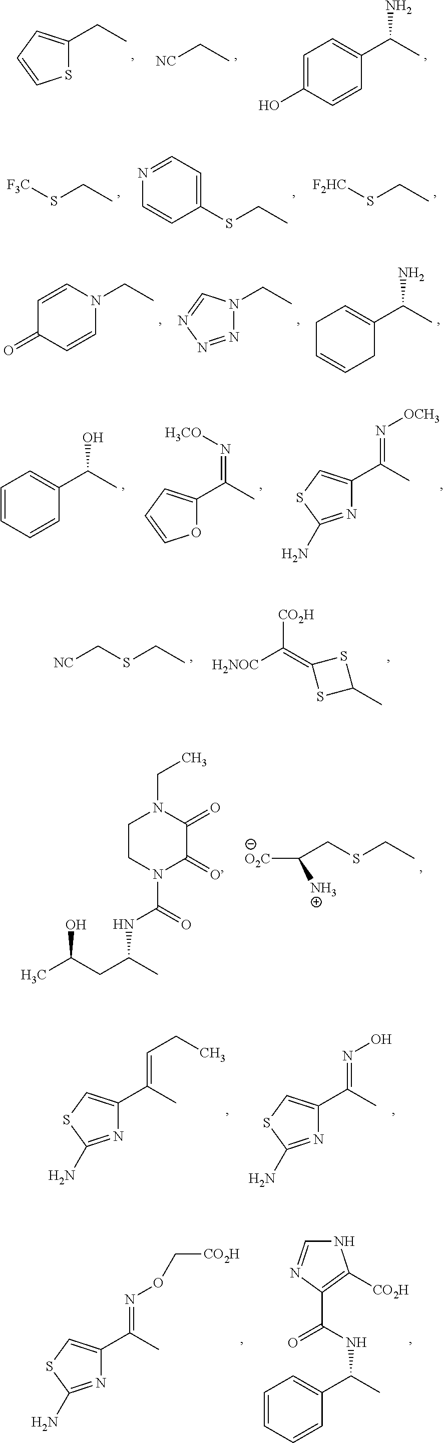

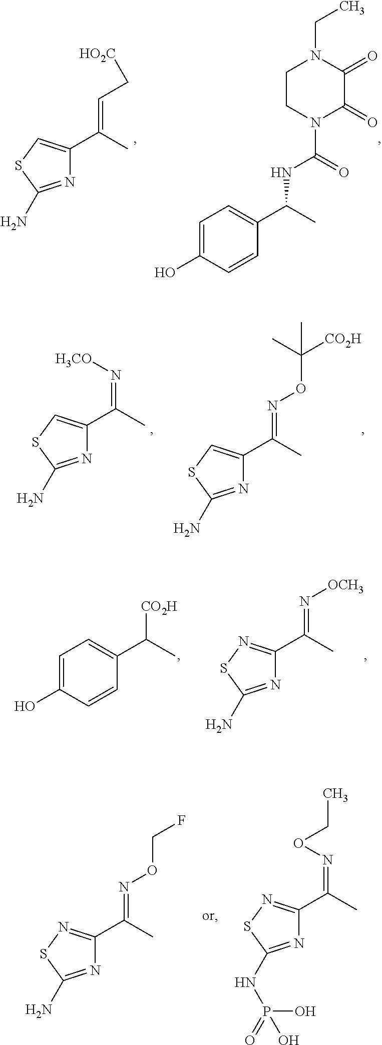

It will be understood that variations and modifications in R2 are permitted so long as they do not significantly impair the ability of the compound to interact with and inhibit the β-lactamase enzyme.

R³ is CR⁸R⁹Z, wherein R⁸ is H or —C₁-C₅ alkyl, R⁹ is H or —C₁-C₅ alkyl, and Z is selected from —S—C(═O)R¹⁰, —S—C(═S)R¹⁰, —SR¹⁰, —SOR¹⁰, —SO₂R¹⁰ or R¹⁰ where R¹⁰ is selected from aryl, preferably having 6 or more carbons, heteroaryl, preferably having 6 or more ring atoms, —(CH₂)ₙ-aryl (where n=1 to 5), —(CH₂)ₙ-heteroaryl (where n=1 to 5), —O(CH₂)ₙH (where n=1 to 5), —S(CH₂)ₙH (where n=1 to 5), —O(CH₂)ₙaryl (where n=0 to 5), —S(CH₂)ₙaryl (where n=0 to 5), —O(CH₂)ₙheteroaryl (where n=0 to 5) or —S(CH₂)ₙheteroaryl (where n=0 to 5), or —O—NHC(═O)R¹¹, —N(SO₂R¹¹)₂ or —OSO₂R¹¹ wherein R¹¹ is selected from —C₁-C₅ alkyl, —CF₃, —CCl₃, aryl, heteroaryl, —(CH₂)ₙ-aryl (wherein n=1 to 5), —(CH₂)ₙ-heteroaryl (wherein n=1 to 5), —O(CH₂)ₙH (wherein n=1 to 5), —S(CH₂)ₙH (wherein n=1 to 5), —O(CH₂)ₙaryl (wherein n=0 to 5), —S(CH₂)ₙaryl (wherein n=0 to 5), —O(CH₂)ₙheteroaryl (wherein n=0 to 5) or —S(CH₂)ₙheteroaryl (wherein n=0 to 5),

In some embodiments, alkyl, aryl and/or heteroaryl may be independently substituted or unsubstituted.

Included herein are also pharmaceutically acceptable salts and esters of the compounds. Skilled persons will be well aware of the scope and meaning of this inclusion.

In some embodiments, Z is —S—C(═O)R¹⁰, —S—C(═S)R¹⁰, —SR¹⁰, —SOR¹⁰, —SO₂R¹⁰ or R¹⁰ where R¹⁰ is as defined above. In some embodiments, R⁸ and R⁹ are each H, and Z is —SC(═O)R¹⁰ or —SC(═S)R¹⁰, and R¹⁰ is preferably aryl having 6 or more carbons or heteroaryl having 6 or more ring atoms. In some preferred embodiments, Z is —SC(═O)R¹⁰. Such compounds have been shown to be particularly good β-lactamase inhibitors. In some embodiments, R¹⁰ is substituted aryl or heteroaryl. In some embodiments, aryl or heteroaryl is independently substituted at, e.g. 1, 2, 3 or 4 positions, with, for example but not limited to, alkyl, preferably lower alkyl, carboxy, carboalkoxy, carboxamido, acyl, aryl, heteroaryl, halo, haloalkyl, haloalkoxy, hydroxy, alkyl, heteroalkyl, aryl, heteroaryl, alkoxy, thioalkoxy, amino, alkylamino, amido, cyano, nitro, oxo, carbonyl, alkoxycarbonyl, thiocarbonyl, acyl, formyl, sulfonyl, mercapto, alkylthio, alkyloxy, alkylamino, cycloalkyl, heterocycloalkyl, aryl, heteroaryl, carboxy, amino, alkylamino, dialkylamino, carbamoyl, aryloxy, heteroaryloxy, arylthio, or heteroarylthio. In some embodiments, R¹⁰ is unsubstituted heteroaryl. In some embodiments, R¹⁰ is unsubstituted aryl. In some embodiments, aryl is phenyl, which may be substituted or unsubstituted as defined herein.

In some embodiments, R³ is a methlybenzoylthio radical:

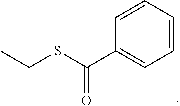

In some embodiments, R² is substituted or unsubstituted —CH₂-aryl or —CH₂-heteroaryl, such as unsubstituted —CH₂-aryl. In some embodiments, R² is substituted —CH₂-aryl. Aryl may be substituted at one or more positions with any suitable substituent. In some embodiments, aryl is substituted with halogen. In some embodiments, halogen is F or Cl. The halogen may be positioned anywhere on the aryl, for example, at C1, C2, C3, C4, C5,or C6 of a phenyl ring, when aryl is phenyl. In some embodiments, the halogen is in the para position on a phenyl ring. In some embodiments, R² is substituted —CH₂-aryl wherein aryl is substituted with —OC₁-C₅ alkyl. In some embodiments, the —OC₁-C₅ alkyl is OCH₃. In some embodiments, the —OCH₃ is in the meta position on a phenyl ring. In some embodiments, R² is —C(═N—OR^{′″})-aryl or —C(═N—OR)-heteroaryl, wherein aryl and heteroaryl may be substituted or unsubstituted. In some embodiments, R² is —C(═N—OR^{′″})-heteroaryl. In some embodiments, R^{′″} is lower alkyl, such as —CH₃. In some embodiments, the heteroaryl portion of the substituent is aminothiazole, such as 2-aminothiazole, or thiophene. The heteroaryl substituents may be attached to the β-lactam at any suitable position on the substituent. In some embodiments, the heteroaryl group is attached as shown below:

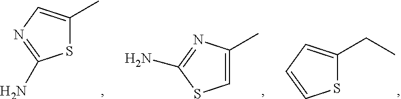

where the line extending from the substituent represents the point of attachment to the rest of the molecule. The same is true for other substituents shown by structure herein.

In some embodiments, R2 is a member of the group shown below:

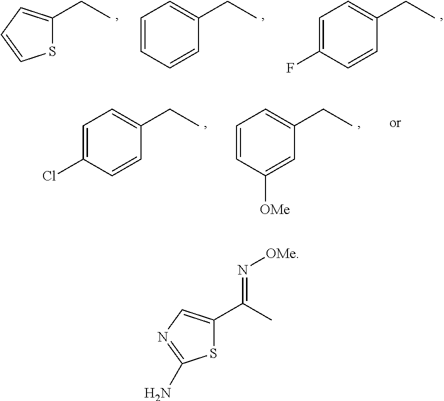

In some embodiments, R² is selected from the group of radicals found on known cephalosporin antibiotics, as in the list presented above.

Any suitable combination of R² and R³, each independently as defined above, is contemplated herein. Particularly preferred are embodiments where R³ is of a size and shape suitable to induce a conformation change in the β-lactamase enzyme which results in inhibition of the enzyme to prevent hydrolysis of a β-lactam antibiotic.

In some embodiments, there is provided a compound of Formula (I):

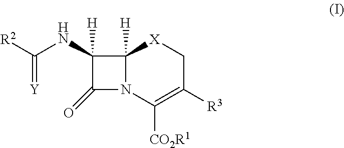

wherein X is O or S; Y is O or S; R¹ is selected from H and a pharmaceutically acceptable cation; R² and R³ are as defined above, wherein alkyl, aryl and heteroaryl may be substituted or unsubstituted; or a pharmaceutically acceptable salt thereof, with the proviso that the compound is not a previously known cephalosporin antibiotic, such as, ceftiofur, moxalactam, cefaloridin or cefalonium or any other cephalosporin antibiotic disclosed herein.

In some embodiments of the compound above, R¹⁰ is:

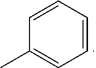

In some embodiments of the compound above, R² is selected from one of the radicals found in cephalosporin antibiotics, as presented above. In some embodiments of the compound above, R² is:

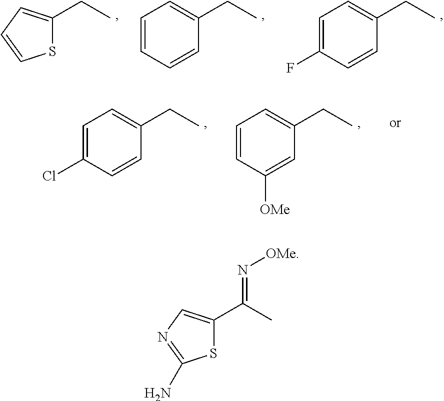

In another aspect, there is provided a compound of Formula (II)

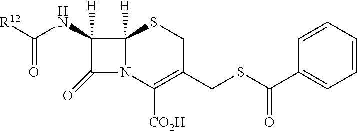

wherein R¹² is as defined broadly above for R² of Formula (I).

In some embodiments of the compound above, R¹² is selected from a radical found in a cephalosporin antibiotic, such as those presented in the list above.

In some embodiments of Formula (II), R¹² is:

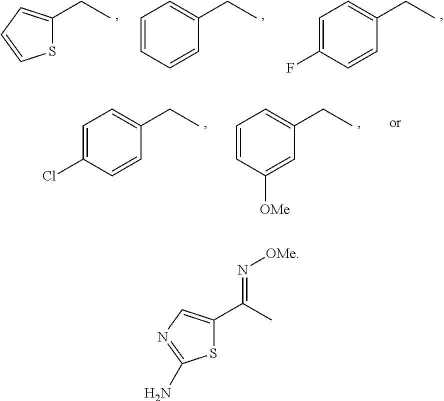

In another aspect, there is provided a compound selected from the group consisting of:

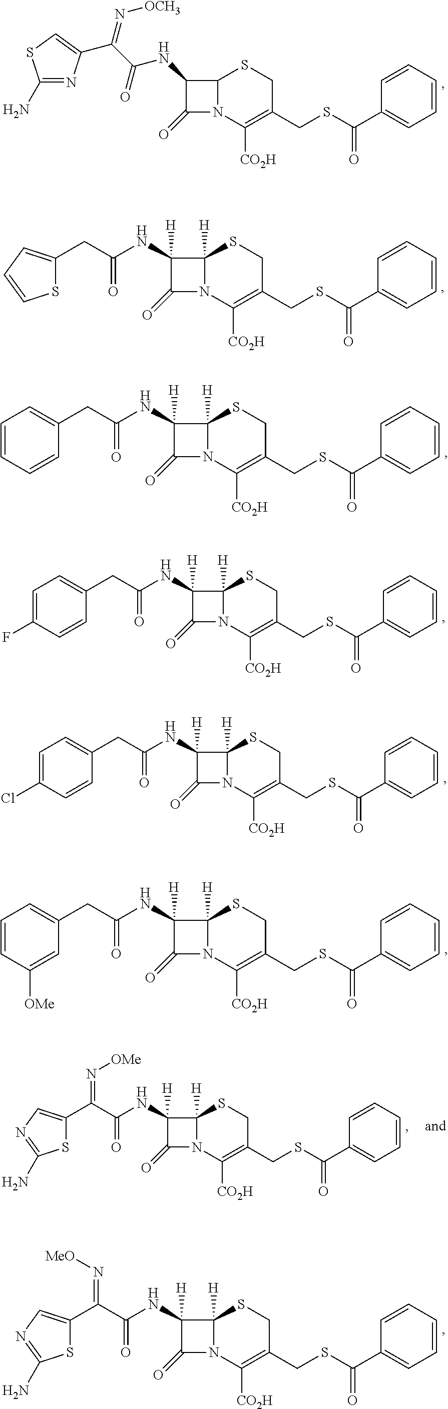

or a pharmaceutically acceptable salt or ester thereof.

In some embodiments, the compound is a C3-benzoylthiomethyl derivative (which may also be called a 3′-benzoylthio derivative) of a cephalosporin antibiotic. It has been found that C3-benzoylthiomethyl derivatives are effective inhibitors of β-lactamases, more than the parent antibiotic, although their antibiotic effects are generally weaker. Although a few benzoylthio derivatives of cephalosporin have been made in past (see, e.g. U.S. Pat. No. 3,243,435), such compounds have never made it to market because they were investigated as antibiotics and were found to have weak antibiotic activity. Such compounds were not examined for β-lactamase inhibition.

The C3-benzoylthiomethyl derivatives have the general Formula (II):

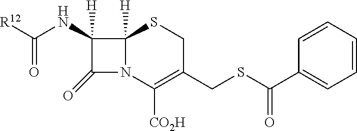

where R¹² is a side group corresponding to a cephalosporin antibiotic. It will be recognized that the class of cephalosporin antibiotics continues to grow. Radicals from new cephalosporin antibiotics, which do not impair the interaction of the compound with β-lactamase, and inhibition thereof, are contemplated within the scope of the present disclosure.

The current cephalosporin antibiotics are divided into several generations as noted below.

First generation cephalosporins include, for example, Cefamandole, Cefazolin, Ceforanide, Cefacetrile (cephacetrile), Cefadroxil (cefadroxyl; Duricef), Cephalexin (cephalexin; Keflex), Cefaloglycin (cephaloglycin), Cefalonium (cephalonium), Cefaloridine (cephaloradine), Cefalotin (cephalothin; Keflin), Cefapirin (cephapirin; Cefadryl), Cefatrizine, Cefazaflur, Cefazedone, Cefazolin (cephazolin; Ancef, Kefzol), Cefradine (cephradine; Velosef), Cefroxadine, Ceftezole. Thus, in some embodiments, the compound is a C3-benzoylthiomethyl derivative of any one of the above.

The second generation cephalosporins include, for example, Cefradine, Cefaclor (Ceclor, Distaclor, Keflor, Raniclor), Cefonicid (Monocid), Cefprozil (cefproxil; Cefzil), Cefuroxime (Zefu, Zinnat, Zinacef, Ceftin, Biofuroksym, Xorimax), Cefuzonam, Cefmetazole, Cefotetan, Cefoxitin. The following cephems are also sometimes grouped with second-generation cephalosporins: Carbacephems: loracarbef (Lorabid); Cephamycins: cefbuperazone, cefmetazole (Zefazone), cefminox, cefotetan (Cefotan), cefoxitin (Mefoxin). Thus, in some embodiments, the compound is a C3-benzoylthiomethyl derivative of any one of the above.

Third generation cephalosporins include, for example, Cefpodoxime, Ceftriaxone, Cefcapene, Cefdaloxime, Cefdinir (Zinir, Omnicef, Kefnir), Cefditoren, Cefetamet, Cefixime (Ziff, Suprax), Cefmenoxime, Cefodizime, Cefotaxime (Claforan), Cefovecin (Convenia), Cefpimizole, Cefpodoxime (Vantin, PECEF), Cefteram, Ceftibuten (Cedax), Ceftiofur, Ceftiolene, Ceftizoxime (Cefizox), Ceftriaxone (Rocephin). Third-generation cephalosporins with antipseudomonal activity: Cefoperazone (Cefobid), Ceftazidime (Fortum, Fortaz). The following cephems are also sometimes grouped with third-generation cephalosporins: Oxacephems: latamoxef (moxalactam).Thus, in some embodiments the compound is a C3-benzoylthiomethyl derivative of any one of the above.

Third forth generation cephalosporins include, for example, Cefclidine, Cefepime (Maxipime), Cefluprenam, Cefoselis, Cefozopran, Cefpirome (Cefrom), Cefquinome. The following cephems are also sometimes grouped with fourth-generation cephalosporins: Oxacephems: flomoxef. Thus, in some embodiments, the compound is a C3-benzoylthiomethyl derivative of any one of the above.

Third forth generation cephalosporins include, for example, Ceftobiprole, Ceftaroline.

Contemplated equivalents of the compounds described above include compounds which otherwise correspond thereto, and which have the same general properties thereof (e.g., precursors, solvates, hydrates), or wherein one or more simple variations of substituents are made which do not adversely affect the efficacy of the compound to function as a β-lactamase inhibitor.

It will be understood that Formula (I) encompasses compounds of Formula (II) as defined herein as well.

It has been found that compounds of formula (I) which are C3-benzoylthiomethyl derivatives of cephalosporins are particularly useful as β-lactamase inhibitors. Surprisingly and importantly, such compounds were shown to be potent inhibitors of the Class B metallo-β-lactamases, although inhibition of the serine β-lactamases was also found.

In some embodiments, the compound is used to broadly inhibit one or more of a Class A, B, C or D β-lactamase. It will be understood that a given compound may have more activity against one or more of the classes compared to others. β-lactamase inhibition can be determined by methods known to those skilled in the art, for example, a β-lactamase inhibition assay (e.g. see Examples). In some embodiments, the compound inhibits one or more of the serine-β-lactamases, e.g. Class A, C and D β-lactamases. In some embodiments, the compound inhibits Class A β-lactamases. In some embodiments, the compound inhibits Class C β-lactamases. In some embodiments, the compound inhibits Class D β-lactamases.

In some embodiments, the compound inhibits one or more metallo-β-lactamases, e.g. Class B β-lactamases. The metallo-β-lactamases may include, for example, IMP-1, VIM-2, or NDM-1. Inhibition of Class B β-lactamases is of particular interest because these enzymes catalyze the hydrolysis of almost all β-lactam antibiotics, including the carbapenems which serve as antibiotics of last resort.

Thus, the compounds disclosed herein are useful in preventing or treating bacterial resistance to an antibiotic, in particular, a β-lactam antibiotic which is sensitive to hydrolysis by β-lactamases. The compounds are particularly effective when administered to a patient in combination with an antibiotic. By “administered in combination with”, or the like, it is meant that the inhibitor compound is administered such that it will exert β-lactamase inhibitory activity (e.g. inhibit β-lactamase) while the antibiotic is active in the patient's system. It will be understood that the inhibitor and the antibiotic need not necessarily be administered at the same time or in the same composition.

Without being bound by theory, it is believed that the compounds disclosed herein bind to β-lactamase enzyme but are relatively poor substrates for the enzyme (e.g.

result in slow turnover of the enzyme), compared to an effective antibiotic with which they may be administered. By binding to and inhibiting the β-lactamase enzyme, the compound protects the antibiotic from hydrolysis by β-lactamase.

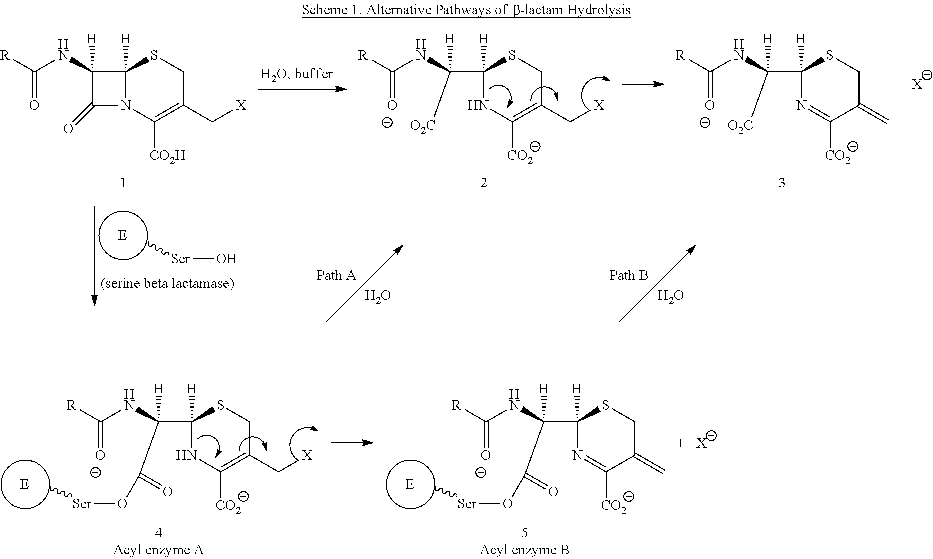

Referring to Scheme 1 above, it is known that the cephalosporoic acid 2, formed upon hydrolysis of the beta lactam bond of a cephalosporin 1, can undergo an elimination reaction to generate a species such as 3 with the expulsion of X⁻ if X is a sufficiently good leaving group. This process occurs both in the spontaneous hydrolysis of a cephalosporin in aqueous medium or in the hydrolysis of the cephalosporin catalyzed by a β-lactamase. This phenomenon has been studied in some detail by Pratt and co-workers (Faraci, W. S.; Pratt, R. F., 1985, Biochemistry, 24, 903-910, Faraci, W. S.; Pratt, R. F 1986, Biochemistry, 25, 2934-2941)

As an example, with the Class A serine beta lactamase PC1 from Staphylococcus aureus, the reaction initially forms acyl enzyme 4 that then is partitioned between two pathways. Pathway A is the normal second stage of the acyl enzyme mechanism that involves enzyme catalyzed hydrolysis of the acyl enzyme to produce the cephalosporoic acid 2 and to regenerate the catalytically active form of the serine β-lactamase. Pathway B involves an alternative elimination reaction wherein the X⁻ group is expelled from the acyl enzyme. It has been shown that the acyl enzyme B undergoes some form of conformational change such that catalysis of the hydrolysis of the acyl enzyme is impaired. The indicated mechanism cannot be operational in the metallo-β-lactamases because of the absence of a nucleophilic serine residue in the active site of MBLs. Thus, results from inhibition studies of serine-β-lactamases cannot necessarily be extrapolated to the MBLs.

More recently, in vitro inhibition of the MBL, CfiA, from Aeromonas hydrophila by moxalactam and by cefoxitin was reported by Galleni and co-workers. (Zervosen, A.; Valladares, M. H.; Devreese, B.; Propsperi-Meys, C.; Adolph, H.-W.; Mercuri, P. S.; Vanhove, M.; Amicosante, G.; van Beeumen, J.; Frère, J.-M.; Galleni, M., 2001, Eur. J. Biochem., 268, 3840-3850.) However, the inhibition was found to require high concentrations of the cephalosporins (>1 mM) and to be irreversible and slow, e.g. full inactivation by moxalactam required 14 hours of incubation. The inhibition process described was specific to the CfiA MBL, which differs from the other know MBLs in that it lacks a second zinc ion and has an exposed cysteine residue in the active site which, in the other MBLs, serves as a ligand for the second zinc ion. Inhibition by the indicated mechanism was not expected for the other MBLs nor was it observed. In some embodiments herein, the MBL is other than CfiA.

Compounds disclosed herein inhibit metallo-β-lactamases in a reversible fashion, unlike the irreversible inhibition observed for the CfiA MBL described above with cefotaxime and moxalactam. In some embodiments, the compounds possess an amide or thioamide linage at the C7 nitrogen atom with a side chain that leads to fairly tight binding in the Michaelis complex with the enzyme.

In preferred embodiments, the compounds incorporate a good leaving group at the C3′ carbon that can be readily expelled as an anion. In some embodiments, the leaving group is a C3′-benzoylthio group, which may be substituted or unsubstituted, according to the definitions provided above for substituted or unsubstituted. In the case of cephalosporins with a benzoylthio group at C3′, it has been demonstrated herein that inhibition of hydrolysis of the chromogenic substrate nitrocefin is observed with all classes of β-lactamases (serine Class A, C and D and metallo Class B). Interestingly, the inhibitory effect is most prominent with metallo-β-lactamases (see Table 1).

That the benzoylthio group is involved in inhibition is revealed by the fact that a Compound A [(6R,7R)-3-(benzoylthiomethyl)-8-oxo-7-(2-(thiophen-2-yl)acetamido)-5-thia-1-azabicyclo[4.2.0]oct-2-ene-2-carboxylic acid from Example 13] is a good inhibitor whereas an oxygen analogue without the thio moiety is a substrate but not an inhibitor. This effect correlates with the expected better leaving group ability of the benzoylthio group in the hydrolysis product versus the benzoate group in the comparative compound. In the case of the metallo-β-lactamases, the possibility that the benzoylthio ion might be the inhibiting species was discounted by the finding that pure benzoylthio is a poor inhibitor of the MBL IMP-1. For compound A, it has been found that incubation of IMP-1 with this inhibitor increases the sensitivity of the protein to proteolysis by trypsin suggesting that the conformation of the enzyme is altered in some way as a consequence of interaction with the inhibitor.

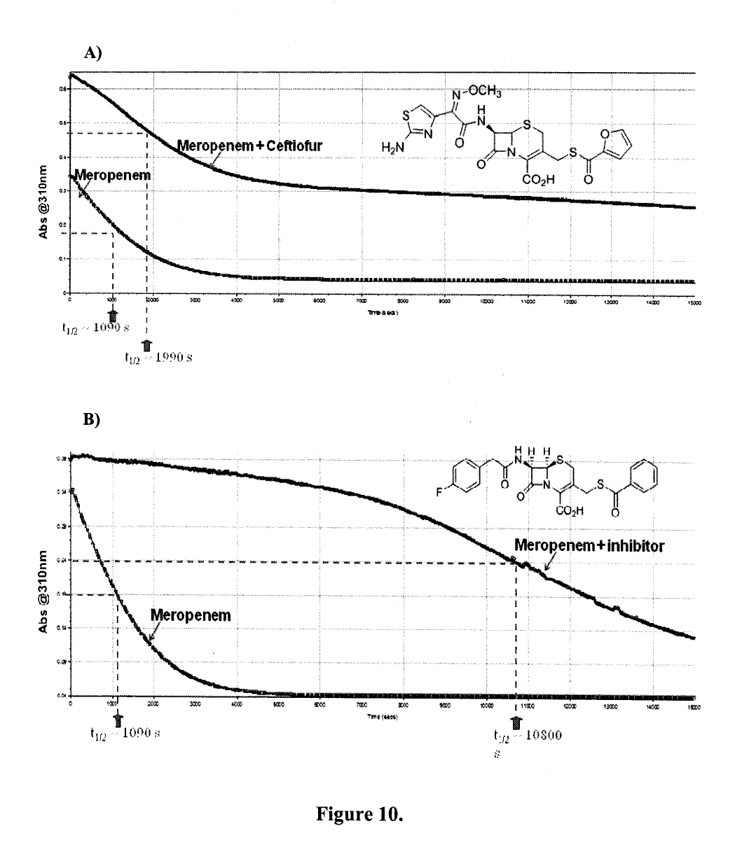

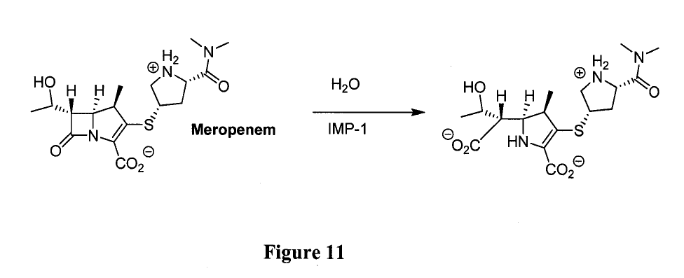

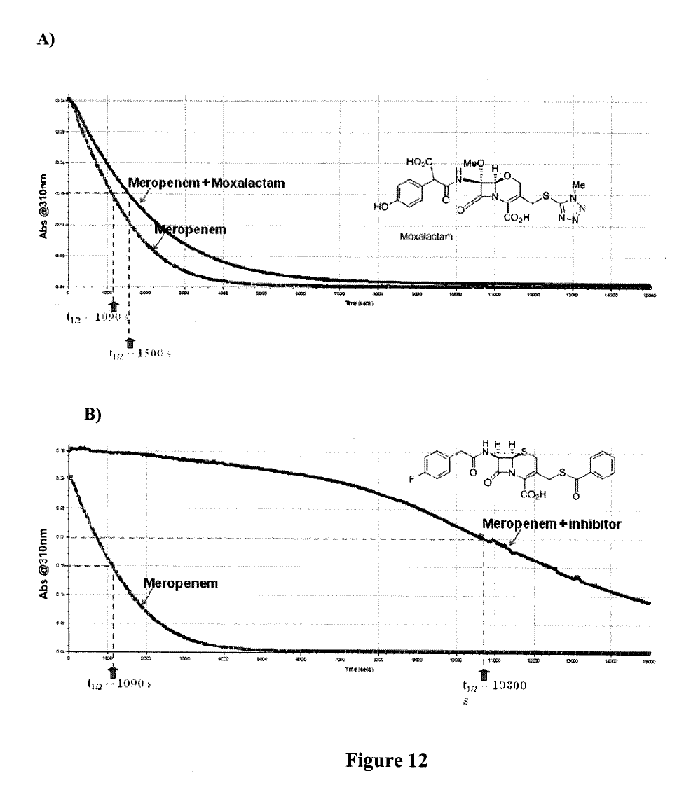

FIG. 10 demonstrates that an inhibitor compound disclosed herein is an effective agent for inhibition of IMP-1 since it extends the half-life of meropenem by a factor of about 10. On the other hand, ceftiofur is a comparatively weak inhibitor of IMP-1 since under comparable conditions it extends the half-life of meropenem by a factor of only 1.7. Likewise FIG. 12 shows that moxalactam is a very weak inhibitor of IMP-1 and that it extends the half-life of meropenem by a factor of only 1.4.

Referring to Scheme 2 below, it is believed that, for the compounds disclosed herein, binding of the compound in the Michaelis complex 17 induces a conformational change in the protein to a state, 18, that binds the compound more tightly. As a result of the affinity of the compound for this conformational state of the enzyme, the initial hydrolysis product has a sufficiently long lifetime so that it eliminates the leaving group at C3′ while still bound to the active site (19→20) and the elimination product, then serves as a relatively potent binding agent for the two active site zinc ions.

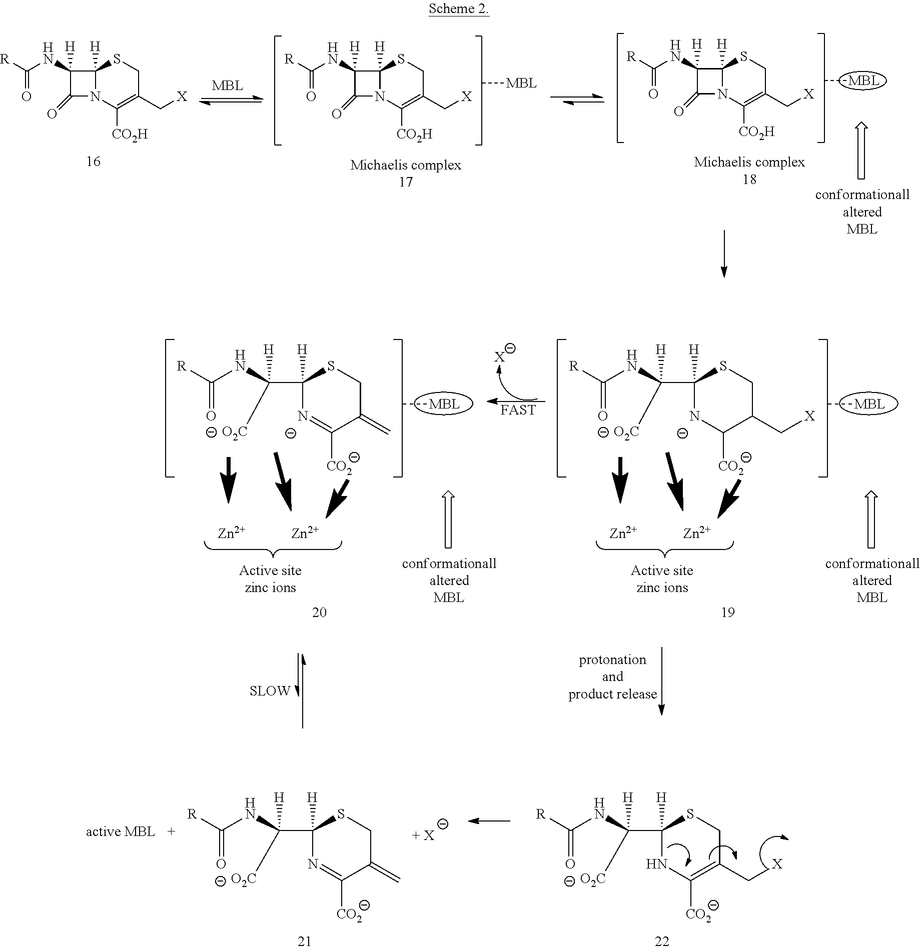

The poor performance of ceftiofur, moxalactam and the thioacetyl derivative are believed to be a consequence of weak interactions of the C3′ with the active site amino acid residues that result in no conformational change of the enzyme to a state that has higher affinity for the compound. Without being bound by theory, the comparatively larger substituents at C3′ of the compounds disclosed herein may play a role in inducing the conformation change in the protein that is essential for the observed inhibition.

Table 2 provides the results of in vitro antimicrobial assays with MBL-producing clinical isolates that are resistant to carbapenems revealing that compounds of this invention can act to enhance the potency of the carbapenem meropenem in a synergistic fashion.

It has been found that a combination of a compound of formula (I), and antibiotic, in particular, a β-lactam antibiotic, shows a synergistic antimicrobial (e.g. antibacterial) effect that significantly exceeds the additive antimicrobial effects of the two compounds. In some embodiments, the inhibitor compound may exhibit some antibiotic activity on its own. However, in some embodiments, the inhibitor compound exhibits little to no antibiotic activity but significantly enhances the activity of the antibiotic. Enhancement in antibiotic activity may be measured in a number of ways known to those skilled in the art, for example, prolongation of antibiotic half life (T_{1/2}) and/or reduction in minimum inhibitory concentration (MIC) of the antibiotic and/or reduction in 50% Inhibition Concentration (IC₅₀) of the antibiotic, compared to the activity of the antibiotic in the absence of the inhibitor compound.

In some embodiments, the inhibitor compound prolongs the half life (T_{1/2}) of the antibiotic by up to about 20 times compared to the half life of the antibiotic in the absence of the inhibitor. In some embodiments, the half life of the antibiotic is prolonged up to about 1.5, 2, 3, 4, 5, 6, 7, 8, 9, 10, 11, 12, 13, 14 15, 16, 17, 18, 19 or 20 times. In some embodiments, the inhibitor prolongs the half life by about 1.5 to about 20 times, e.g. about 1.5 to about 15 times, about 1.5 to about 10 times, about 5 to about 10 times, about 5 to about 15 times, about 10 to about 20 times, or about 5 to about 20 times. In some embodiments, the inhibitor compound prolongs the half life (T_{1/2}) of the antibiotic in vitro. The prolongation of antibiotic activity is expected to occur in vivo as well since the β-lactam antibiotics are hydrolyzed by β-lactamases in vivo. Furthermore, β-lactamase inhibitors are generally known to prolong the activity of β-lactam antibiotics.

In some embodiments, the inhibitor reduces the minimum inhibitory concentration (MIC) of the antibiotic by up to about 99% times compared to MIC of the antibiotic in the absence of the inhibitor. In some embodiments, the MIC is reduced up to about 10%, 20%, 30%, 40%, 50%, 60%, 70%, 80%, 90%, 95%, 98% or 99%, compared to the MIC of the antibiotic in the absence of the inhibitor. In some embodiments, the inhibitor compound reduces the MIC of the antibiotic in vitro.

In one aspect, there are provided pharmaceutical compositions comprising a compound of Formula (I) and a pharmaceutically acceptable excipient or carrier. In some embodiments, there is provided a pharmaceutical composition for preventing or treating bacterial resistance to an antibiotic, the composition comprising a β-lactamase inhibitory amount of a compound as defined herein, and a pharmaceutically acceptable excipient.

“Pharmaceutically acceptable excipient” means an excipient that is useful in preparing a pharmaceutical composition and that is compatible with the other ingredients in the composition, and one that is generally safe, non-toxic and neither biologically nor otherwise undesirable. The term includes excipients that are acceptable for veterinary use as well as human pharmaceutical use. A “pharmaceutically acceptable excipient” as used in the specification and claims includes both one and more than one such excipient.

A “β-lactamase inhibitory amount” is an amount required to achieve a desired outcome that is a result of inhibition of β-lactamase, e.g. inhibition of β-lactamase activity itself, enhanced antibiotic activity, reduction in symptoms or underlying cause of an infection when the inhibitor is administered together with an antibiotic, etc. A skilled person will be able to determine the appropriate amount.

In some embodiments, the pharmaceutical composition comprises a pharmaceutically acceptable antibiotic, in particular, a β-lactam antibiotic. Any suitable β-lactam antibiotic may be selected, including those recited anywhere herein. Examples include a penicillin, a cephalosporin, an oxacephem, a carbacephem, a cephamycin, an oxacephamycin, a penem, or a carbapenem. In some embodiments, the β-lactam antibiotic is a carbapenem, such as, imipenem, meropenem, ertapenem, doripenem, panipenem/betamipron, biapenem, or razupenem (PZ-601).

In some embodiments, the β-lactamase to be inhibited is a metallo-β-lactamase. Examples of metallo-β-lactamases are provided elsewhere herein.

The compounds and/or pharmaceutical compositions described herein may be used in the manufacture of a medicament for treating a bacterial infection and/or preventing or treating bacterial resistance to an antibiotic. That is to say, the compounds and compositions are for use in the manufacture of such a medicament.

The compounds and/or pharmaceutical compositions described herein may be used in treating a bacterial infection and/or preventing or treating bacterial resistance to an antibiotic. In some embodiments, the compounds and/or pharmaceutical compositions described herein may be used for treating a nosocomial bacterial infection.

The bacterial infection may be caused by a bacteria expressing at least one β-lactamase enzyme, e.g. a class A, B, C or D β-lactamase. In some embodiments, the at least one β-lactamase enzyme is a metallo-β-lactamase. In some embodiments, the β-lactamase is a carbapenemase. Examples of metallo-β-lactamases are known to those skilled in the art and are disclosed elsewhere herein.

The compounds of formula (I) can be formulated in pharmaceutical compositions by combining the compounds with a pharmaceutically acceptable excipient. Examples of such excipients are set forth below. The compounds of formula (I) have β-lactamase inhibitory properties, and are useful when combined with a β-lactam antibiotic for the treatment of infections in animals, especially mammals, including humans. The compounds may be used, for example, in the treatment of infections of the respiratory tract, urinary tract and soft tissues and blood, among others.

The compositions of the invention include those in a form adapted for administration by a variety of means: for instance, orally, topically, or parenterally by injection (such as intravenously, intramuscularly, or subcutaneously). The compounds of formula (I), may be employed in powder or crystalline form, in liquid solution, or in suspension.

Suitable forms of the compositions of this invention include tablets, capsules, creams, syrups, suspensions, solutions, emulsions in oily or aqueous vehicles, reconstitutable powders and sterile forms suitable for injection or infusion. The pharmaceutical compositions may contain conventional pharmaceutically acceptable excipients such as buffering agents, diluents, binders, excipients, colours, flavours, preservatives, disintegrants and the like in accordance with conventional pharmaceutical practice in the manner well understood by those skilled in the art of formulating antibiotics.

In injectable compositions, for instance, the carrier may be typically comprised of sterile water, saline, or another injectable liquid. Solutions of the compounds of formula (I) can be prepared in water, optionally mixed with a nontoxic surfactant. Dispersions can also be prepared in oils, and in glycerol, liquid polyethylene glycols, triacetin, and mixtures thereof. Under ordinary conditions of storage and use, these preparations typically contain a preservative to prevent the growth of microorganisms. Injectable solutions may be sterilized by incorporating the compound of formula (I) in the required amount in an appropriate solvent, with various other ingredients which may be desired, and filter sterilizing the resulting solution. Where sterile powders are needed, preferred methods of preparing these powders are vacuum drying and freeze-drying sterile solutions of the compounds of formula (I),) in combination with other desired ingredients.

Topical compositions may be formulated in various excipients. Such excipients may be hydrophobic or hydrophilic bases to form ointments, creams, lotions, in aqueous, oleaginous or alcoholic liquids to form paints or in dry diluents to form powders. Useful solid excipients include finely divided solids such as talc, clay, microcrystalline cellulose, silica, alumina and the like. Useful liquid excipients include water, alcohols or glycols or water-alcohol/glycol blends, in which the compounds of formula (I) can be dissolved or dispersed at effective levels, optionally with the aid of non-toxic surfactants. Fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers. Thickeners known to those of skill in the art, such as synthetic polymers, fatty acids or salts and esters thereof, fatty alcohols, etc. can be used with liquid excipients to form spreadable pastes, gels, ointments, soaps, etc.

The following references disclose useful dermatological compositions which can be used to deliver the compounds of formula (I) to the skin: Jacquet et al. (U.S. Pat. No. 4,608,392), Geria (U.S. Pat. No. 4,992,478), Smith et al. (U.S. Pat. No. 4,559,157), and Wortzman (U.S. Pat. No. 4,820,508).

Oral compositions may be in the form of oral solutions or suspensions, or may be in tablet or capsule form (such as hard or soft shell gelatine capsules). Oral compositions include both extended release and immediate release delivery forms. Compositions for oral administration may also be incorporated directly with the food of a patients diet. The compounds of formula (I), may be combined with one or more excipients and used in the form of ingestible tablets, buccal tablets, troches, capsules, elixirs, suspensions, syrups, wafers, and the like. Such compositions and preparations should contain at least 0.1% of active compound. The amount of the compounds of formula (I) in such therapeutically useful compositions is such that an effective dosage level will be obtained.

The above-mentioned compositions for oral administration may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, etc. may be added. When the unit dosage form is a capsule, it may contain a liquid carrier, such as a vegetable oil or a polyethylene glycol, in addition to materials of the above type. Various other materials may be present as coatings, etc. For instance, tablets, pills, or capsules may be coated with gelatin, wax, shellac or sugar and the like. A syrup or elixir may contain the active compound, a sweetening agent, one or more preservatives, a dye and a flavouring agent. Of course, any material used in preparing any unit dosage form should be pharmaceutically acceptable and substantially non-toxic in the amounts employed.

The compounds of formula (I) may be present in the composition as sole active agents or may be present together with other therapeutic agents such as a pharmaceutically acceptable β-lactam antibiotic. It is generally advantageous to use a compound of formula (I) in admixture or conjunction with a carbapenem, penicillin, cephalosporin or other β-lactam antibiotic or prodrug. It may also be advantageous to use a compound of formula (I) in combination with one or more β-lactam antibiotics, because of the β-lactamase inhibitory properties of the compounds. In this case, the compound of formula (I) and the β-lactam antibiotic can be administered separately or in the form of a single composition containing both active ingredients.

In some embodiments, the concentration of the compound(s) of formula (I) in a liquid composition, such as a lotion, or a semi-solid or solid composition, such as a gel or a powder, will be from about 0.1-99 wt %, or from about 0.5-50 wt %, or 0.5-25 wt %, for liquid compositions and 0.1-15 wt % or 0.1-5 wt % for semi-solid or solid compositions. When the compositions are presented in unit dosage form, each unit dose may suitably comprise from about 10 to about 1500 mg, or about 25 to about 1000 mg of a compound of formula (I). although lower or higher doses may be used in accordance with clinical practice. Appropriate dosages of the compounds of formula (I) may be readily ascertained by those of skill in the art.

When the compounds of formula (I) are co-administered with a β-lactam antibiotic, the ratio of the amount of the compounds of formula (I) to the amount of the β-lactam antibiotic may vary within a wide range. In some embodiments, the ratio may, for example, be from 1:100 to 100:1. The amount of β-lactam antibiotic will normally be approximately similar to the amount in which it is conventionally used. In some cases, it may be necessary to titrate the dose of the antibiotic since the activity may be enhanced when administered to the patient together with the inhibitor.

In some embodiments, between about 25 and 6000 mg of the compositions of the invention, for example, may be administered each day of treatment, although such daily dosages may be readily ascertained by those of skill in the art. For the treatment of severe systemic infections or infections of particularly intransigent organisms, higher doses may be used in accordance with clinical practice.

In another aspect, there is provided a method of treating a bacterial infection and/or preventing or treating bacterial resistance to an antibiotic comprising administering to a patient in need thereof a β-lactamase inhibitory amount of a compound of formula (I) as defined above in combination with a therapeutically effective amount of an antibiotic, e.g. pharmaceutically acceptable antibiotic. A therapeutically effective amount of an antibiotic is an amount which is effective for treating or reducing the symptoms or underlying cause of the bacterial infection. In some embodiments, the patient is a mammalian patient, such as a mammalian animal or human.

In some embodiments, the antibiotic is a β-lactam antibiotic, including but not limited to, a penicillin, a cephalosporin, an oxacephem, a carbacephem, monobactams, a cephamycin, an oxacephamycin, a penem, or a carbapenem or other pharmaceutically acceptable β-lactam antibiotic. Pharmaceutically acceptable β-lactam antibiotics suitable for co-administration with the compounds of formula (I), whether by separate administration or by inclusion in the compositions according to the invention, include both those known to show instability to or to be otherwise susceptible to β-lactamases and also known to have a degree of resistance to β-lactamases. Well known β-lactam antibiotics have been described by Selwyn and Essack ((a) Selwyn, S. J. Antimicrob. Chemother. 1982, 9 (Suppl. B), 1-10; (b) Essack, S. Y. Pharm. Res. 2001, 18, 1391-1399). Exemplary antibiotics for use in accordance with the compositions, methods, packages and kits disclosed herein include but are not limited to those examples listed below and elsewhere in the application.

Examples of carbapenems that may be co-administered with the compounds of formula (I) include imipenem, meropenem, biapenem and doripenem (4R,5S,6S)-3-[(3S,5S)-5-(3-carboxyphenyl-carbamoyl)pyrrolidin-3-ylthio]-6-(1R)-1-hydroxyethyl]-4-methyl-7-oxo-1-azabicyclo[3.2.0]hept-2-ene-2-carboxylic acid, (1S,5R,6S)-2-(4-(2-(carbamoylmethyl)-1,4-diazoniabicyclo[2.2.2]oct-1-yl)-ethyl(1,8-naphthosultam) methyl)-6-[1(R)-hydroxyethyl]-1-methyl carbapen-2-em-3-carboxylate chloride, BMS181139 ([4R-[4α,5β,6β(R*)]]-4-[2-[(aminoiminomethyl)amino]ethyl]-3-[(2-cyanoethyl)thio]-6-(1-hydroxyethyl)-7-oxo-1-azabicyclo[3.2.0]hept-2-ene-2-carboxylic acid), B02727 ([4R-3[3S*,5S*(R*)], 4α,5β,6β(R*)]]-6-(1-hydroxyethyl)-3-[[5-[1-hydroxy-3-(methylamino)propyl]-3-pyrrolidinyl]thio]-4-methyl-7-oxo-1-azabicyclo[3.2.0]hept-2-ene-2-carboxylic acid monohydrochloride), E1010 ((1R,5S,6S)-6-[1(R)-hydroxymethyl]-2-[2(S)-[1(R)-hydroxy-1-[pyrrolidin-3(R)-yl]methyl]pyrrolidin-4(S)-yl sulfanyl]-1-methyl-1-carba-2-penem-3-carboxylic acid hydrochloride), S4661 ((1R,5S,6S)-2-[(3S,5S)-5-(sulfamoylaminomethyl)pyrrolidin-3-yl]thio-6-[(1R)-1-hydroxyethyl]-1-methylcarbapen-2-em-3-carboxylic acid) and (1S,5R,6S)-1-methyl-2-{7-[4-(aminocarbonylmethyl)-1,4-diazoniabicyclo[2.2.2]octan-1-yl]-methyl-fluoren-9-on-3-yl}-6-(1R-hydroxyethyl)-carbapen-2-em-3-carboxylate chloride and (+)-(4R,5S,6S)-6-[(1R)-1-1hydroxyethyl]-4-methyl-7-oxo-3 [[3S,5S)—S-(sulfamoylaminomethyl)pyrrolidin-3-yl]thio]-1-azabicyclo[3.2.0]hept-2-ene-2-carboxylic acid monohydrate.

Certain carbapenems (e.g. imipenem) are susceptible to destruction by human renal dehydropeptidase (DHP); thus, pharmaceutical compositions comprising compounds of formula (I) and such carbapenems may further comprise an inhibitor for DHP, such as cilastatin. In some embodiments, the β-lactam antibiotic is a carbapenem, such as, Imipenem, Meropenem, Ertapenem, Doripenem, Panipenem/betamipron, Biapenem, Razupenem (PZ-601).

Examples of penicillins suitable for co-administration with the compounds of formula (I) include benzylpenicillin, phenoxymethylpenicillin, carbenicillin, azidocillin, propicillin, ampicillin, amoxycillin, epicillin, ticarcillin, cyclacillin, pirbenicillin, azlocillin, mezlocillin, sulbenicillin, piperacillin, and other known penicillins. The penicillins may be used in the form of prodrugs thereof; for example as in vivo hydrolysable esters, for example the acetoxymethyl, pivaloyloxymethyl, a-ethoxycarbonyloxy-ethyl and phthalidyl esters of ampicillin, benzylpenicillin and amoxycillin; as aldehyde or ketone adducts of penicillins containing a 6-α-aminoacetamido side chain (for example hetacillin, metampicillin and analogous derivatives of amoxycillin); and as a-esters of carbenicillin and ticarcillin, for example the phenyl and indanyl α-esters.

Examples of cephalosporins include, cefatrizine, cephaloridine, cephalothin, cefazolin, cephalexin, cephacetrile, cephapirin, cephamandole nafate, cephradine, 4-hydroxycephalexin, cephaloglycin, cefoperazone, cefsulodin, ceftazidime, cefuroxime, cefinetazole, cefotaxime, ceftriaxone, ceftazidime, ceftabiprole, ceftaroline fosamil, and other known cephalosporins, such as those recited elsewhere above, all of which may be used in the form of prodrugs thereof.

Examples of β-lactam antibiotics other than penicillins and cephalosporins that may be co-administered with the compounds of formula (I) include aztreonam, latamoxef (Moxalactam™), and other known β-lactam antibiotics such as carbapenems like imipenem, meropenem or (4R,5S,6S)-3-[(3S,5S)-5-(3-carboxyphenylcarbamoyl)pyrrolidin-3-ylthio]-6-(1R)-1-hydroxyethyl]-4-methyl-7-oxo-1-azabicyclo[3.2.0]hept-2-ene-2-carboxylic acid, all of which may be used in the form of prodrugs thereof

The methods disclosed herein may be used to treat a bacterial infection involving a bacteria that expresses at least one β-lactamase enzyme, e.g. a β-lactamase enzyme from any of the classes of β-lactamase. In some embodiments, β-lactamase enzyme is a metallo-β-lactamase. Thus, in some embodiments, there is provided a method of inhibiting a metallo-β-lactamase or a method of treating an infection involving a bacteria that produces a metallo-β-lactamase. In some embodiments, the bacterial infection to be treated is a nosocomial infection or a hospital-acquired infection.

In another aspect, there is provided a method of inhibiting a β-lactamase enzyme, the method comprising contacting the β-lactamase enzyme with a compound of formula (I) as defined above. The β-lactamase inhibition may occur in vitro or in vivo.

Exemplary bacteria to be targeted include, but are not limited to, a Gram positive bacteria such as methicillin-resistant Staphylococcus aureus (MRSA); methicillin-susceptible Staphylococcus aureus (MSSA); glycopeptide intermediate-susceptible Staphylococcusaureus (GISA); methicillin-resistant Staphylococcus epidermitis (MRSE); methicillin-sensitive Staphylococcus epidermitis (MSSE); vancomycin-sensitive Enterococcus faecalis (EFSVS); vancomycin-sensitive Enterococcus faecium (EFMVS); penicillin-resistant Streptococcus pneumoniae (PRSP); Streptococcus pyogenes; Bacillus anthracia and Gram negative bacteria such as Salmonella enterica; Salmonella typhi; Shigella dysenteriae; Yersinia pestis; Pseudomonas aeruginosa; Vibrio cholerae; Bordetalla petussis; Haemophilus injiuenzae; Helicobacter pylori; Helicobacterfells; Campylobacter jejuni; Neisseria gonorrhoeae; Neisseria meningitides; Brucellaabortus; Bacteroides fragilis; Acinetobacter baumannii; Chryseobacterium meningosepticum; Stenotrophomonas maltophilia; Serratia marscesens; Burkholderia cepacia, or Acinetobacter baumannii.

In some embodiments, the bacteria is an organism that is commonly targeted by carbapenems in nosocomial infections and that produces carbapenemase (e.g Pseudomonas aeruginosa,).

In another aspect, there is provided a commercial package or kit comprising a compound or a composition as defined herein, together with instructions for use in inhibiting a β-lactamase and/or preventing or treating bacterial resistance to an antibiotic and/or treating a bacterial infection.

Those of skill in the art will appreciate that useful dosages of the compounds of formula (I) can be determined by comparing their in vitro activity, and in vivo activity in animal models. Methods for the extrapolation of effective dosages in mice, and other animals, to humans are known to the art; for example, see U.S. Pat. No. 4,938,949.

The compounds disclosed herein are derivatives of the cephalosporins (cephems X═S; oxacephems X═O, aza cephem X═NR and carbacephems X═C). As a class, the cephalosporin derived antibacterial compounds have been in clinical use for more than 30 years. The properties of relatively low toxicity and high bioavailability that is characteristic of such compounds can be reasonably expected to be observed with compounds of the present disclosure.

The cephalosporins are largely excreted via the urine and hence caution is normally recommended for patients with renal insufficiency. This will likely be the case with medicaments involving the compounds of the present disclosure. The cephalosporins and likely the compounds of this disclosure are contraindicated with patients with known allergic reaction to other beta lactam antibiotics.

Common adverse drug reactions (≧1% of patients) associated with the cephalosporin therapy include: nausea, diarrhea, electrolyte disturbances, rash, and pain and inflammation at the injection site. It is reasonable to expect that related adverse reactions might be observed with compounds of the present invention in clinical applications. Other properties of the first, second, third, fourth and fifth generation cephalosporins are described more fully in the Merck Manuals On-line Medical Library (http://www.merckmanuals.com/professional/sec14/ch170/ch170c.html)

In general, the compounds of the present disclosure may be prepared by the methods illustrated in the reaction schemes disclosed herein, for example, as described above, or by modifications thereof, using readily available starting materials, reagents and conventional synthesis procedures. In some embodiments, the compounds are prepared as illustrated in the Figures and the Examples. In these reactions, it is also possible to make use of variants which are in themselves known, but are not mentioned. It is noted that many of the starting materials employed in the synthetic methods described herein are commercially available or are reported in the scientific literature.

The articles “a” and “an” are used herein to refer to one or to more than one (i.e., to at least one) of the grammatical object of the article. By way of example, “an element” means one element or more than one element.

The terms “comprise” and “comprising” are used in the inclusive, open sense, meaning that additional elements may be included.

The term “including” is used to mean “including but not limited to”. “Including” and “including but not limited to” are used interchangeably.

### EXAMPLES

### Example 1

### Enzyme Preparation

Compounds of the present application were evaluated as inhibitors of the class A β-lactamase KPC-2, the class B β-lactamases IMP-1,VIM-2, and NDM-1 the class C β-lactamase GC1, and the class D β-lactamases OXA-10 and OXA-45.

The KPC-2 β-lactamase was purified from Escherichia coli DH5α strain containing the plasmid, pBR322-catI-bla_{KPC-2}, which was kindly provided by Prof. F. van den Akker (Case Western Reserve University). Expression and purification of the enzyme was similar to that reported by Ke et al. (Biochemistry 2007, 46, 5732-5740). A molecular weight of 28,472 Da for the purified KPC-2 was determined by ESI MS. This is in close agreement with that calculated for a mature protein with a 24 amino acid signal peptide removed.

IMP-1 metallo-β-lactamase was expressed in Esherichia coli BL21(DE3) carrying pCIP4 and purified as reported by Laraki et al. (Antimicrob. Agents Chemother. 1999, 43, 902-906). The homogeneity of the protein was confirmed by SDS-PAGE according to the method of Laemmli (Nature 1970, 227, 680-685). The molecular weight of purified IMP-1, Mᵣ=25,112±0.5 Da, as determined by ESI-MS, was in agreement with that deduced from its amino acid sequence (Laraki et al. Antimicrob. Agents Chemother. 1999, 43, 890-901).

VIM-2 metallo-β-lactamase was expressed in Escherichia coli BL21 (DE3) pLysS carrying pNOR2001 (a generous gift from P. Nordmann, Université Paris XI, France) and purified as reported by Poirel et al. (Antimicrob. Agents Chemother. 2000, 44, 891-897). The homogeneity and molecular weight of the mature VIM-2 (29.7 kDa) was determined by SDS-PAGE (Laemmli, U. K. Nature 1970, 227, 680-685). Enzyme concentration was determined from the absorbance at 280 nm (εEM=32,000 M⁻¹ cm⁻¹).

GC1 β-lactamase was purified from Escherichia coli AS226-51 harbouring pCS100, which was a generous gift from Prof. M. Nukaga (Josai International University, Japan). The cell culture was harvested and subjected to a stringent periplasmic lysis protocol according to Crichlow et al. (Biochemistry 1999, 38, 10256-10261.). Purification proceeded similarly to Nukaga et al. (J. Biol. Chem. 2004, 279, 9344-9352.) with cation exchange chromatography (CM Sephadex C-50) equilibrated with 10 mM sodium phosphate buffer (pH 6), followed by elution with a stepwise gradient of increasing NaCl concentration. Fractions containing β-lactamase activity were then applied to a phenylboronate column (MoBiTec, Germany), equilibrated with 20 mM triethanolamine.HCl, pH 7.0, 0.5 M NaCl. The column was washed with the same buffer and then eluted with sterile 0.5 M borate buffer, pH 7.0, 0.5 M NaCl. Active fractions eluted from the phenylboronate column were analyzed for purity via SDS-PAGE, pooled, and concentrated before buffer exchange to 100 mM sodium phosphate, pH 7.0.

OXA-10 β-lactamase was expressed and purified using a modified protocol of that reported by Golemi et al. (J. Am. Chem. Soc. 2000, 122, 6132-6133). The pET24a plasmid with the bla_{OXA-10} gene insert, a kind gift from Prof. S. Mobashery (Notre Dame University), was transformed into Escherichia coli BL21 (DE3) and induced with IPTG. Purification of the enzyme was achieved by the addition of ammonium sulfate to the induced culture supernatant. The protein precipitate collected (50-70% ammonium sulfate saturation) was dissolved and dialyzed against 10 mM Tris.SO₄ buffer, pH 7.5, and further purified with a DE52 cellulose column (Whatman, UK) equilibrated with the same buffer. The material bound to the column was washed with 10 mM Tris.SO₄ buffer, pH 7.5, followed by an elution with a K₂SO₄ gradient (0-100 mM). Protein fractions containing nitrocefin-hydrolyzing activity were concentrated and purity analyzed by SDS-PAGE. A molecular weight of 27,490 Da for OXA-10 was determined by ESI MS, indicating a post-translational deletion of a 20 amino acid signal peptide.

OXA-45 β-lactamase was purified in the laboratory of Prof. T. Walsh (University of Bristol, UK) according to the method published (Toleman, M. A.; Rolston, K.; Jones, R. N.; Walsh, T. R. Antimicrob. Agents Chemother. 2003, 47, 2859-2863) and provided to this laboratory as a kind gift.

### Example 2

### Inhibition of β-Lactamases

Assay conditions for IC₅₀ determinations involved enzyme concentrations ranging between 2 and 6 nM. The concentration used for the nitrocefin substrate was 100 μM. Assays with OXA-10 and OXA-45 were conducted at pH 7.0 with 100 mM sodium phosphate buffer containing 25 mM sodium bicarbonate. Assays with KPC-2 and GC1 were performed at pH 7.0 in 50 mM HEPES buffer and assays with IMP-1 and VIM-2 were performed in the same buffer with the addition of 500 mM NaCl and 1 μM ZnSO₄. BSA was used with all enzymes (except OXA-10 and KPC-2) at concentrations of 0.5-1.0 μg/mL.

Inhibitors were pre-incubated for 10 minutes at room temperature with enzyme assay solutions prior to the addition of the nitrocefin. These compounds were dissolved in DMSO and prepared at various concentrations such that the final concentration of DMSO was 1% during the pre-incubation. The reaction was initiated by the addition of the pre-incubated enzyme-cyclobutanone solution (90 μL) to a solution of nitrocefin in buffer (10 μL). Enzymatic activity was determined using the initial rate of increase in absorbance at 482 nm

The results are summarized in Table 1.

TABLE 1
Inhibition of serine-β-lactamases (Class A, C and D) and metallo-β-
lactamases (Class D) by a 3′-thiobenzoyl cephalosporin derivative.^{a}

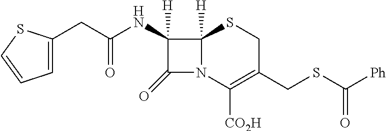

| Class A | Class B | Class B | Class B | Class C | Class D | Class D |
|---|---|---|---|---|---|---|
| KPC-2 | IMP-1 | VIM-2 | NDM-1^{b} | GC1 | OXA-10 | OXA-45 |
| 71 μM | 3.1 μM | 1.8 μM | 33 μM | 140 μM | 8.1 μM | 24 μM |

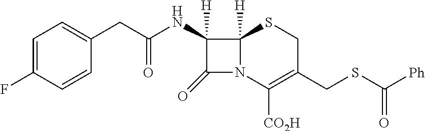

| Class A | Class B | Class B | Class B | Class C | Class D | Class D |
|---|---|---|---|---|---|---|
| KPC-2 | IMP-1 | VIM-2 | NDM-1^{b} | GC1 | OXA-10 | OXA-45 |
| 104 μM | 8.2 μM | 4.2 μM | 40 μM | 11 μM | 6.3 μM | n.d.^{c} |

^{a}IC₅₀ values
^{b}NDM-1 conditions included 15 μM nitrocefin.
^{c}n.d. = not determined

### Example 3

### Antimicrobial Assays

For the cryopreservation of microorganisms, porous beads in sterile vials were used to serve as excipients to support microorganisms (purchased from Microbank, Pro-Lab Diagnostics). A loopful of culture was taken from an agar plate and added to the vial containing cryopreservative fluid and beads. The vial was shaken and excess cryopreservative aspirated, leaving the beads as free of liquid as possible. The vial was stored at −80° C. Recovery of the microorganism involves the transfer of a single bead from the vial into media in a test tube while the remaining beads are returned to −80° C. to prevent them from thawing.

Assays were conducted as described by Weigand et al. (Weigand, I.; Hilpert, K.; Hancock, R. E. Nat. Protocols 2008, 3, 163-175).

MIC Determinations

To determine the lowest concentration of an antimicrobial agent that inhibits visible growth of a microorganism, 50 μL of compound solution and 50 μL of buffer solution were added to each well at the desired concentration(s) (i.e. 4×the final concentration) as well as a zero control. Stock solutions of 10 mg/mL, were prepared in DMSO, followed by two-fold serial dilutions in Müeller-Hinton media and 100 μL of bacteria (ca. 5×10⁵ CFU/mL) was added to each well. Plate counts were performed on the control wells to determine bacterial innoculum level. The innoculum was prepared by taking an overnight culture in Müeller-Hinton media and diluting to McFarland's 0.5 standard, and diluting a further 1:100 prior to addition to the plate. Lanes with no bacteria added served as sterility controls and lanes with no compound served as growth controls. Organisms were allowed to grow overnight at 37° C. in a humid environment and the plates were monitored at 625 nm for growth. MIC was determined to be the last well with no growth.

Checkerboard Assays for Synergy

In the checkerboard assay one compound is serially diluted along the ordinate, and the second compound is serially diluted along the abscissa. Meropenem (50 μL) was added to each well in a dilution series from 512 μg/mL to 1 μg/mL in 100 mM phosphate buffer pH 6.8 (i.e. 4×the final concentration), including a zero control. A 50 μL aliquot of the compound was added to each well at the desired concentration(s) (i.e. 4×the final concentration) as well as a zero control. Stock solutions of 10 mg/mL of compound were prepared in DMSO, followed by two-fold serial dilutions in Müeller-Hinton media. Finally, 100 μL of bacteria (ca. 5×10⁵ CFU/mL) was added to each well. Plate counts were performed on the control wells. Innoculum was prepared by taking an overnight culture in Müeller-Hinton media and diluting to McFarland's 0.5 standard, and diluting a further 1:100 prior to addition to the plate. Lanes with no bacteria added served as sterility controls and lanes with no meropenem or compound served as growth controls. The final volume in each well was 200 μL. The plates were allowed to grow overnight at 37° C. in a humid environment and were monitored at 625 nm for growth. The MIC is determined as the lowest concentration that completely inhibits visible growth of an organism. Sterility controls and growth controls were included on each plate. The fractional inhibitory concentration (FIC) was determined as follows:

$$FIC = \frac{Lowest MIC ( A combination )}{Lowest MIC ( A alone )} + \frac{Lowest MIC ( B combination )}{Lowest MIC ( B alone )}$$

Results were interpreted in the following way: Synergistic if FIC≦0.5; Additive if 0.5<FIC>1.0; Indifferent if 1.0<FIC>2.0; Antagonistic if FIC≧2.0.

TABLE 2
Antimicrobial activity of meropenem alone, and in the presence of two inhibitors,
against carbapenem-resistant MBL-producing clinical isolates.^{a}

| | |
|---|---|
| 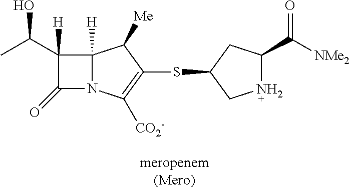 | |
| | CMPD A |
| 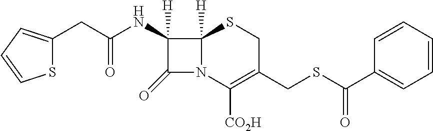 | |
| | CMPD B |
| 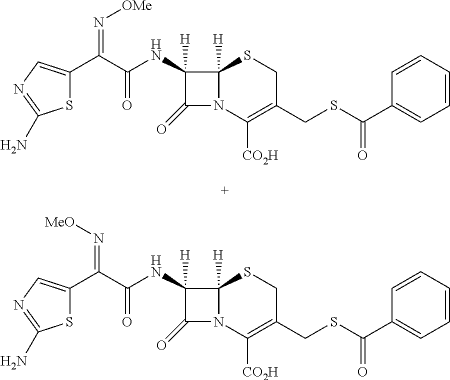 | |
| | CMPD C |
| 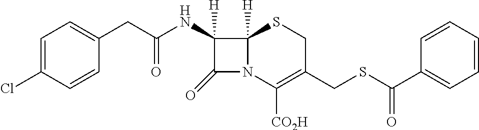 | |
| | CMPD D |
| 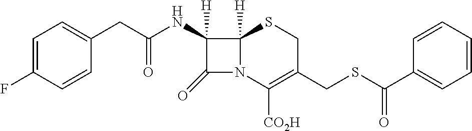 | |
| | CMPD E |
| 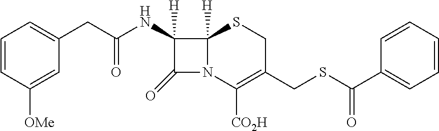 | |
| | CMPD F |
| 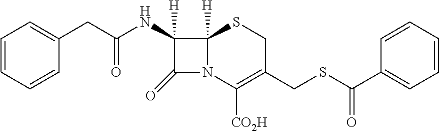 | |

| | | MIC (μg/mL) | MIC (μg/mL) | |
|---|---|---|---|---|
| | MIC (μg/mL) | Mero + CMPD A | Mero + CMPD A | |
| Clinical Isolate^{a} | Mero Alone^{b} | (100 μg/mL) | (25 μg/mL) | FIC |
| Pseudomonas aeruginosa C-10 | 16 | 8 | 16 | 0.5625 |
| Pseudomonas aeruginosa IS 5563 | 64 | 4 | 32 | 0.1289 |
| Stenotrophomonas maltophilia IS 5568 | 64 | 4 | 16 | 0.1328 |
| Chryseobacterium meningosepticum IS 5824 | 64 | <.25 | 32 | 0.2539 |
| Stenotrophomonas maltophilia IS 6069 | 64 | 16 | 32 | 0.25 |
| Stenotrophomonas maltophilia IS 6081 | 32 | <.25 | 16 | 0.2539 |
| Pseudomonas aeruginosa IS 6225 | 2 | 1 | 1 | 0.625 |
| | | MIC (μg/mL) | MIC (μg/mL) | |
| | MIC (μg/mL) | Mero + CMPD B | Mero + CMPD B | |
| Clinical Isolate^{a} | Mero Alone^{b} | (100 μg/mL) | (25 μg/mL) | FIC |
| Pseudomonas aeruginosa C-10 | | | | |
| Pseudomonas aeruginosa IS 5563 | 64 | 1 | 32 | 0.1328 |
| Stenotrophomonas maltophilia IS 5568 | 64 | 4 | 32 | 0.1289 |
| Chryseobacterium meningosepticum IS 5824 | 32 | <.25 | 32 | 0.5078 |
| Stenotrophomonas maltophilia IS 6069 | 64 | 8 | 16 | 0.1875 |
| Stenotrophomonas maltophilia IS 6081 | 32 | 2 | 32 | 0.1328 |
| Pseudomonas aeruginosa IS 6225 | 8 | 2 | 2 | 0.375 |
| | | MIC (μg/mL) | MIC (μg/mL) | |
| | MIC (μg/mL) | Mero + CMPD C | Mero + CMPD C | |
| Clinical Isolate^{a} | Mero Alone^{b} | (100 μg/mL) | (25 μg/mL) | FIC |
| Pseudomonas aeruginosa C-10 | 16 | 8 | 16 | 1 |
| Pseudomonas aeruginosa IS 5563 | 128 | <0.25 | 32 | 0.2519 |
| Stenotrophomonas maltophilia IS 5568 | 128 | 1 | 32 | 0.126 |
| Chryseobacterium meningosepticum IS 5824 | 32 | <0.25 | 2 | 0.2578 |
| Stenotrophomonas maltophilia IS 6069 | 4 | <0.25 | 1 | 0.1875 |
| Stenotrophomonas maltophilia IS 6081 | 8 | <0.25 | 2 | 0.281 |
| Pseudomonas aeruginosa IS 6225 | 1 | 1 | 0.5 | 0.625 |
| | | MIC (μg/mL) | MIC (μg/mL) | |
| | MIC (μg/mL) | Mero + CMPD D | Mero + CMPD D | |
| Clinical Isolate^{a} | Mero Alone^{b} | (100 μg/mL) | (25 μg/mL) | FIC |
| Pseudomonas aeruginosa C-10 | 16 | 8 | 8 | 1 |
| Pseudomonas aeruginosa IS 5563 | 128 | <0.25 | 64 | 0.2519 |
| Stenotrophomonas maltophilia IS 5568 | 128 | 16 | 64 | 0.126 |
| Chryseobacterium meningosepticum IS 5824 | 32 | <0.25 | 2 | 0.2578 |
| Stenotrophomonas maltophilia IS 6069 | 4 | <0.25 | 2 | 0.1875 |
| Stenotrophomonas maltophilia IS 6081 | 8 | <0.25 | 8 | 1.03 |
| Pseudomonas aeruginosa IS 6225 | 1 | 1 | 1 | 2 |
| | | MIC (μg/mL) | MIC (μg/mL) | |
| | MIC (μg/mL) | Mero + CMPD E | Mero + CMPD E | |
| Clinical Isolate^{a} | Mero Alone^{b} | (100 μg/mL) | (25 μg/mL) | FIC |
| Pseudomonas aeruginosa C-10 | 16 | 8 | 8 | 0.625 |
| Pseudomonas aeruginosa IS 5563 | 128 | 8 | 32 | 0.1269 |
| Stenotrophomonas maltophilia IS 5568 | 128 | 8 | 32 | 0.126 |
| Chryseobacterium meningosepticum IS 5824 | 32 | 0.25 | 0.5 | 0.2578 |
| Stenotrophomonas maltophilia IS 6069 | 4 | 1 | 1 | 0.1875 |
| Stenotrophomonas maltophilia IS 6081 | 8 | <0.25 | 8 | 1.03 |
| Pseudomonas aeruginosa IS 6225 | 1 | 1 | 1 | 2 |
| | | MIC (μg/mL) | MIC (μg/mL) | |
| | MIC (μg/mL) | Mero + CMPD F | Mero + CMPD F | |
| Clinical Isolate^{a} | Mero Alone^{b} | (100 μg/mL) | (25 μg/mL) | FIC |
| Pseudomonas aeruginosa C-10 | 16 | 16 | 16 | 1.5 |
| Pseudomonas aeruginosa IS 5563 | 128 | 0.5 | 64 | 0.2519 |
| Stenotrophomonas maltophilia IS 5568 | 128 | 16 | 64 | 0.126 |
| Chryseobacterium meningosepticum IS 5824 | 32 | <0.25 | 1 | 0.2578 |
| Stenotrophomonas maltophilia IS 6069 | 4 | <0.25 | 1 | 0.1875 |
| Stenotrophomonas maltophilia IS 6081 | 8 | <0.25 | 4 | 0.281 |
| Pseudomonas aeruginosa IS 6225 | 1 | 2 | 2 | 3 |

^{a}Clinical isolates with IS numbers were isolated from Ontario hospitals and were provided by Prof. D. Pillai (University of Toronto). The P. aeruginosa strain C-10 was isolated from a Calgary hospital and was provided by Prof. J. Pitout (University of Calgary).
^{b}Commercially available meropenem was used as received.

### Example 4

### General Experimental Procedures

Chemical shifts in ¹H NMR and ¹³C NMR spectra are reported in parts per million (ppm) relative to tetramethylsilane (TMS). Calibration of the residual solvent peaks was done according to the values reported by Gottlieb et al. (J. Org. Chem. 1997, 62, 7512-7515. When peak multiplicities are given, the following abbreviations are used: s, singlet; d, doublet; t, triplet; q, quartet; sept., septet; dd, doublet of doublets; m, multiplet; br, broad; app., apparent; gem, geminal. ¹H NMR spectra were acquired at 300 MHz and 500 MHz with a digital resolution (Brüker parameter: FIDRES) of 0.245 and 0.0993 Hz/point, respectively. The coupling constants reported herein therefore have uncertainties of ±0.5 Hz and ±0.2 Hz at 300 MHz and 500 MHz, respectively. Reactions were carried out at room temperature (rt) if temperature is not specified. All reactions were done under an atmosphere of either nitrogen or argon, with the exception of selected reactions done in aqueous media. For the purification of compounds by flash chromatography, 230-400 mesh (40-63 μM) flash silica was used (Silicycle, Québec, QC).

### Example 5

### General Procedure for the Acylation of 7-Aminocephalosporanic Acid.

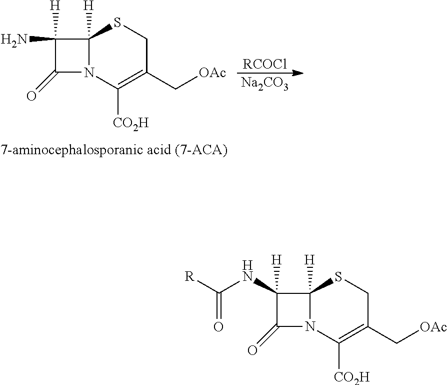

According to the method described by Cocker et al. (Cocker, J. D.; Cowley, B. R.; Cox, J. S. G.; Eardley, S.; Gregory, G. I.; Lazenby, J. K.; Long, A. G.; Sly, J. C. P.; Somerfield, G. A. J. Chem. Soc., 1965, 5015-5031), 7-ACA (272 mg, 1.0 mmol) was added to a stirring solution of Na₂CO₃.H₂O (200 mg, 1.6 mmol) in H₂O (4 mL) at 0° C. This solution was stirred for 5 min at 0° C. before the dropwise addition of the acid chloride (1.1 mmol), followed by the addition of acetone (1 mL). The mixture was stirred for 30 min at 0° C. and then allowed to warm to room temperature overnight. The mixture was diluted with H₂O (40 mL) and adjusted to pH 2 with the addition of 2 N HCl. The resulting precipitate was filtered and dried under vacuum.

### Example 6

### (6R,7R)-3-(acetoxymethyl)-8-oxo-7-(2-phenylacetamido)-5-thia-1 aza bicyclo[4.2.01oct-2-ene-2-carboxylic acid

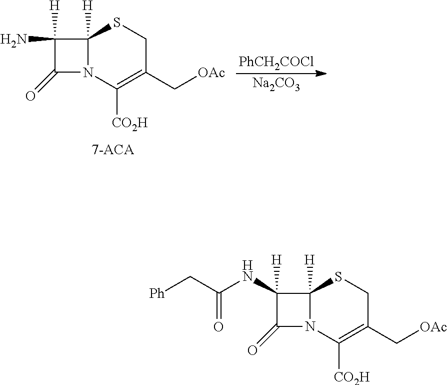

The title compound was isolated as a yellow solid (175 mg, 45%) following the general procedure for acylation described above. ¹H NMR (300 MHz, DMSO-d₆): δ 9.10 (d, J=8.3 Hz, 1H), 7.32-7.18 (m, 5H), 5.67 (dd, J=8.3 Hz, J=4.8 Hz, 1H), 5.07 (d, J=4.8 Hz, 1H), 4.99 (d, J=12.8 Hz, 1H), 4.67 (d, J=12.8 Hz, 1H), 3.61 (d, J=18.1 Hz, 1H), 3.56 (d, J=13.8 Hz, 1H), 3.48 (d, J=13.8 Hz, 1H), 3.47 (d, J=18.1 Hz, 1H), 2.02 (s, 3H). LRMS (+ESI) m/z: 408 [M+H₂O]⁺, 391 [M+H]⁺.

### Example 7

### (6R,7R)-3-(acetoxymethyl)-7-(2-(4-chlorophenyl)acetamido)-8-oxo-5-thia-1-azabicyclo[4.2.0]oct-2-ene-2-carboxylic acid

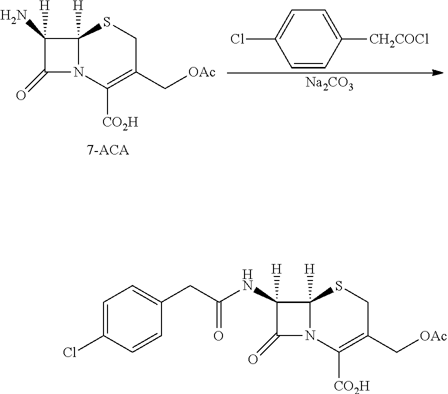

The title compound was isolated as an ivory solid (413 mg, 97%) following the general procedure for acylation described above. ¹H NMR (300 MHz, DMSO-d₆): δ 9.12 (d, J=8.2 Hz, 1H), 7.37-7.25 (m, 4H), 5.66 (dd, J=8.2 Hz, J=4.8 Hz, 1H), 5.07 (d, J=4.8 Hz, 1H), 4.99 (d, J=12.8 Hz, 1H), 4.67 (d, J=12.8 Hz, 1H), 3.61 (d, J=18.2 Hz, 1H), 3.57-3.46 (m, 2H), 3.46 (d, J=18.2 Hz, 1H), 2.02 (s, 3H).

### Example 8

### (6R,7R)-3-(acetoxymethyl)-7-(2-(4-fluorophenyl)acetamido)-8-oxo-5-thia-1-azabicyclo[4.2.0]oct-2-ene-2-carboxylic acid

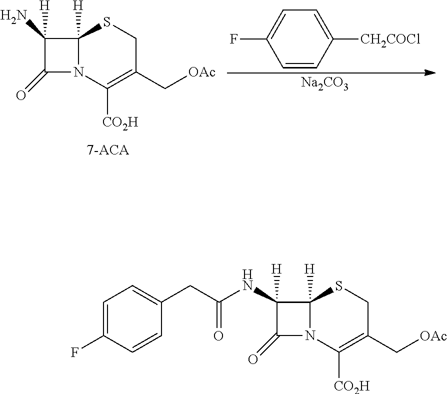

The title compound was isolated as an ivory solid (410 mg, 100%) following the general procedure for acylation described above. ¹H NMR (300 MHz, DMSO-d₆): δ 9.10 (d, J=8.2 Hz, 1H), 7.31-7.26 (m, 2H), 7.14-7.08 (m, 2H), 5.66 (dd, J=8.2 Hz, J=4.8 Hz, 1H), 5.07 (d, J=4.8 Hz, 1H), 4.99 (d, J=12.8 Hz, 1H), 4.67 (d, J=12.8 Hz, 1H), 3.61 (d, J=18.2 Hz, 1H), 3.55 (d, J=14.3 Hz, 1H), 3.48 (d, J=14.3 Hz, 1H), 3.46 (d, J=18.2 Hz, 1H), 2.02 (s, 3H).

### Example 9

### (6R,7R)-3-(Acetoxymethyl)-7-(2-(3-methoxyphenyl)acetamido)-8-oxo-5-thia-1-azabicyclo[4.2.0]oct-2-ene-2-carboxylic acid

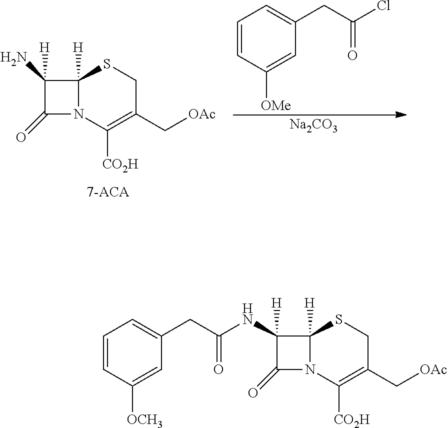

The title compound was isolated as a yellow-beige solid (653.4 mg, 42%) following the general procedure for acylation described above. ¹H NMR (300 MHz, DMSO-d6): δ 13.9-12.5 br s, 9.07 (d, J=8.3 Hz, 1H), 7.19 (t, J=7.8 Hz, 1H), 6.87-6.76 (m, 3H), 5.67 (dd, J=8.3, 4.8 Hz, 1H), 5.07 (d, J=4.8 Hz, 1H), 4.99 (d, J=12.8 Hz, 1H), 4.68 (d, J=12.8 Hz, 1H), 3.73 (s, 3H), 3.69-3.42 (m, 4H), 2.02 (s, 3H).

### Example 10

### (6R,7R)-3-(acetoxymethyl)-8-oxo-7-(2-(thiophen-2-yl)acetamido)-5-thia-1-azabicyclo[4.2.0]oct-2-ene-2-carboxylic acid (Cephalothin)

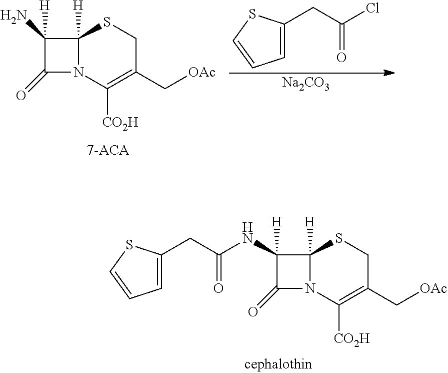

The title compound was isolated as an amber solid (345 mg, 87%) following the general procedure for acylation described above. Spectral data were compatible with that of commercial cephalothin. ¹H NMR (300 MHz, DMSO-d₆): δ 9.12 (d, J=8.1 Hz, 1H), 7.36-7.32 (m, 1H), 6.95-6.91 (m, 2H), 5.68 (dd, J=8.1 Hz, J=4.8 Hz, 1H), 5.08 (d, J=4.8 Hz, 1H), 4.99 (d, J=12.8 Hz, 1H), 4.67 (d, J=12.8 Hz, 1H), 3.77 (d, J=16.0 Hz, 1H), 3.72 (d, J=16.0 Hz, 1H), 3.61 (d, J=18.1 Hz, 1H), 3.50-3.36 (m, 1H), 2.01 (s, 3H). LRMS (−ESI) m/z: 395 [M−H]⁺.

### Example 11

### General Procedure for Substitution at C3′ With Thioacids.

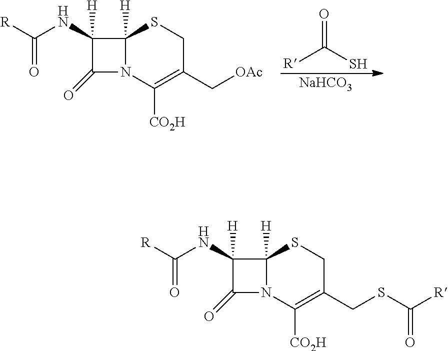

According to the method described by Cowley et al. (Cowley, B. R.; Gregory, G. I.; Lazenby, J. K.; Long, A. G. 7-Acylaminocephalosporanic Acid Derivatives. U.S. Pat. No. 3,243,435, Mar. 29, 1966), the thioacid (4 mmol) was dissolved in a solution of NaHCO₃ (336 mg, 4 mmol) in H₂O (20 mL) at 50° C. This solution was filtered through a pad of silica gel and added to a mixture of the 3′-acetoxycephalosporanic acid derivative (1 mmol) in H₂O (10 mL) at 50° C. and stirred at this temperature for 40 h. The reaction mixture was cooled to rt, diluted with H₂O (60 mL), and the pH was adjusted to 4 with the addition of 6 N HCl. The precipitate was filtered and dried under vacuum.

### Example 12

### (6R,7R)-3-(acetylthiomethyl)-8-oxo-7-(2-(thiophen-2-yl)acetamido)-5-thia-1-azabicyclo[4.2.0]oct-2-ene-2-carboxylic acid

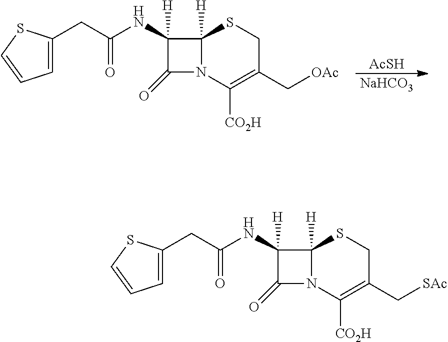

The title compound was isolated as an off-white solid (247 mg, 60%) following the general procedure for substitution described above. ¹H NMR (300 MHz, DMSO-d₆): δ 9.10 (d, J=8.3 Hz, 1H), 7.35-7.33 (m, 1H), 6.94-6.90 (m, 2H), 5.64 (dd, J=8.3 Hz, J=4.8 Hz, 1H), 5.05 (d, J=4.8 Hz, 1H), 4.02 (d, J=13.3 Hz, 1H), 3.75 (d, J=13.3 Hz, 1H), 3.73 (s, 2H), 3.64 (d, J=18.0 Hz, 1H), 3.29 (d, J=18.0 Hz, 1H), 2.33 (s, 3H). LRMS (−ESI) m/z (relative intensity): 411 (M−H)⁺.

### Example 13

### (6R,7R)-3-(benzoylthiomethyl)-8-oxo-7-(2-(thiophen-2-yl)acetamido)-5-thia-1-azabicyclo[4.2.0]oct-2-ene-2-carboxylic acid

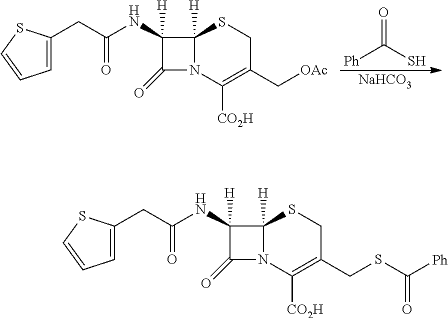

The title compound was isolated as an off-white solid (408 mg, 86%) following the general procedure for substitution described above. ¹H NMR (500 MHz, DMSO-d₆): δ 9.02 (d, J=8.3 Hz, 1H), 7.92-7.90 (m, 2H), 7.67 (app. t, J=7.3 Hz, Hz, 1H), 7.57-7.52 (m, 2H), 7.34 (d, J=4.9 Hz, 1H), 6.94-6.90 (m, 2H), 5.48 (dd, J=8.3 Hz, J=4.7 Hz, 1H), 4.96 (d, J=4.7 Hz, 1H), 4.24 (d, J=12.5 Hz, 1H), 4.13 (d, J=12.5 Hz, 1H), 3.75 (d, J=15.4 Hz, 1H), 3.71 (d, J=15.4 Hz, 1H), 3.54 (d, J=17.4 Hz, 1H), 3.16 (d, J=17.4 Hz, 1H). LRMS (−ESI) m/z (relative intensity): 473 (M−H)⁺.

### Example 14

### (6R,7R)-3-(benzoylthiomethyl)-8-oxo-7-(2-phenylacetamido)-5-thia-1-azabicyclo[4.2.0]oct-2-ene-2-carboxylic acid

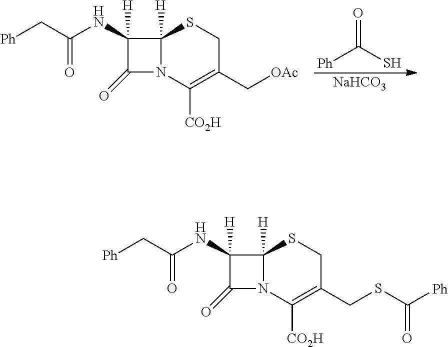

The title compound was isolated as an off-white solid (295 mg, 63%) following the general procedure for substitution described above. ¹H NMR (300 MHz, DMSO-d₆): δ 9.09 (d, J=8.3 Hz, 1H), 7.94-7.90 (m, 2H), 7.70 (br t, J=7.3 Hz, 1H), 7.58-7.52 (m, 2H), 7.30-7.15 (m, 5H), 5.64 (dd, J=8.3 Hz, J=4.8 Hz, 1H), 5.06 (d, J=4.8 Hz, 1H), 4.29 (d, J=13.2 Hz, 1H), 3.99 (d, J=13.2 Hz, 1H), 3.71 (d, J=18.0 Hz, 1H), 3.55 (d, J=13.8 Hz, 1H), 3.46 (d, J=13.8 Hz, 1H), 3.38 (d, J=18.0 Hz, 1H). LRMS (+ESI) m/z: 486 [M+H₂O]⁺, 469 [M+H]⁺.

### Example 15

### (6R,7R)-3-(benzoylthiomethyl)-7-(2-(4-chlorophenyl)acetamido)-8-oxo-5-thia-1-azabicyclo[4.2.0]oct-2-ene-2-carboxylic acid

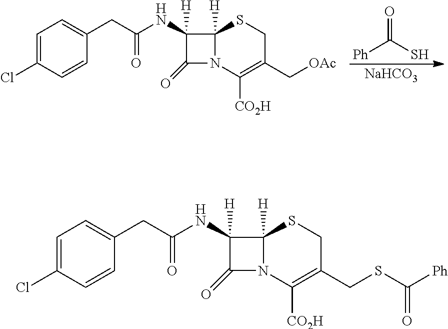

The title compound was isolated as an off-white solid (248 mg, 50%) following the general procedure for substitution described above. ¹H NMR (300 MHz, DMSO-d₆): δ 9.07 (d, J=8.3 Hz, 1H), 7.93-7.90 (m, 2H), 7.69 (br t, J=7.4 Hz, 1H), 7.58-7.53 (m, 2H), 7.35-7.33 (m, 2H), 7.28-7.22 (m, 2H), 5.55 (dd, J=8.3 Hz, J=4.8 Hz, 1H), 5.01 (d, J=4.8 Hz, 1H), 4.26 (d, J=13.0 Hz, 1H), 4.01 (d, J=13.0 Hz, 1H), 3.63 (d, J=17.8 Hz, 1H), 3.55 (d, J=14.0 Hz, 1H), 3.52-3.30 (m, 2H).

### Example 16

### (6R,7R)-3-(benzoylthiomethyl)-7-(2-(4-fluorophenyl)acetamido)-8-oxo-5-thia-1-azabicyclo[4.2.0]oct-2-ene-2-carboxylic acid

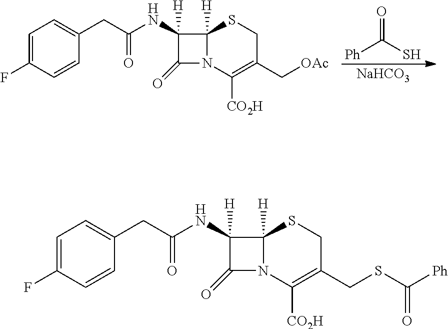

The title compound was isolated as an off-white solid (309 mg, 64%) following the general procedure for substitution described above. ¹H NMR (300 MHz, DMSO-d₆): δ 9.06 (d, J=8.3 Hz, 1H), 7.93-7.90 (m, 2H), 7.69 (br t, J=7.3 Hz, 1H), 7.57-7.52 (m, 2H), 7.30-7.26 (m, 2H), 7.14-7.70 (m, 2H), 5.57 (dd, J=8.3 Hz, J=4.8 Hz, 1H), 5.01 (d, J=4.8 Hz, 1H), 4.27 (d, J=13.1 Hz, 1H), 4.02 (d, J=13.1 Hz, 1H), 3.65 (d, J=17.9 Hz, 1H), 3.53 (d, J=14.0 Hz, 1H), 3.41 (d, J=14.0 Hz, 1H), 3.30 (d, J=17.8 Hz, 1H).

### Example 17

### Sodium (6R,7R)-3-(benzoylthiomethyl)-7-(2-(3-methoxyphenyl)acetamido)-8-oxo-5-thia-1-azabicyclo[4.2.0]oct-2-ene-2-carboxylate

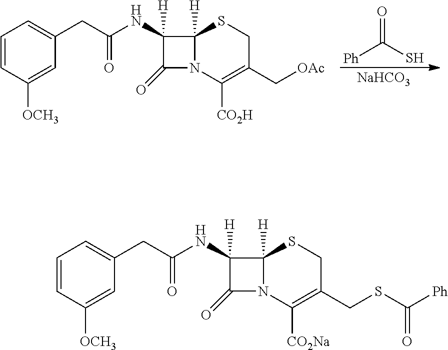

The title compound was isolated as an off-white solid (157.8 mg, 43%) following a modification of the general procedure for substitution described above. The reaction mixture was filtered to isolate the product without prior acidification. ¹H NMR (300 MHz, DMSO-d6): δ 9.03 (d, J=8.4 Hz, 1H), 7.92 (d, J=7.4 Hz, 2H), 7.69 (t, J=7.4 Hz, 1H), 7.55 (t, J=7.4 Hz, 2H), 7.18 (t, J=7.8 Hz, 1H), 6.85-6.73 (m, 3H), 5.57 (dd, J=8.4, 4.8 Hz, 1H), 5.01 (d, J=4.8 Hz, 1H), 4.27 (d, J=13.0 Hz, 1H), 4.05 (d, J=13.0 Hz, 1H), 3.71 (s, 3H), 3.64 (d, J=17.7 Hz, 1H), 3.52 (d, J=13.7 Hz, 1H), 3.43 (d, J=13.7 Hz, 1H), 3.29 (d, J=17.7 Hz, 1H).

### Example 18

### (6R,7R)-3-(benzoylthiomethyl)-7-(2-(4-chlorophenyl)acetamido)-8-oxo-5-thia-1-azabicyclo[4.2.0]oct-2-ene-2-carboxylic acid

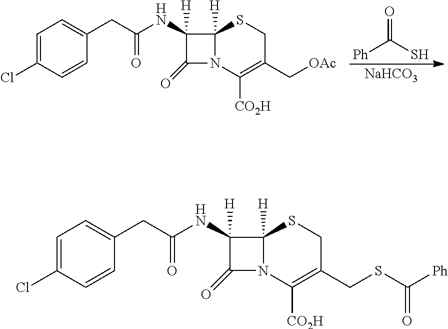

The title compound was isolated as an off-white solid (248 mg, 50%) following the general procedure for substitution described above. ¹H NMR (300 MHz, DMSO-d₆): δ 9.07 (d, J=8.3 Hz, 1H), 7.93-7.90 (m, 2H), 7.69 (br t, J=7.4 Hz, 1H), 7.58-7.53 (m, 2H), 7.35-7.33 (m, 2H), 7.28-7.22 (m, 2H), 5.55 (dd, J=8.3 Hz, J=4.8 Hz, 1H), 5.01 (d, J=4.8 Hz, 1H), 4.26 (d, J=13.0 Hz, 1H), 4.01 (d, J=13.0 Hz, 1H), 3.63 (d, J=17.8 Hz, 1H), 3.55 (d, J=14.0 Hz, 1H), 3.52-3.30 (m, 2H).

### Example 19

### (6R,7R)-3-(benzoylthiomethyl)-7-(2-(4-fluorophenyl)acetamido)-8-oxo-5-thia-1-azabicyclo[4.2.0]oct-2-ene-2-carboxylic acid

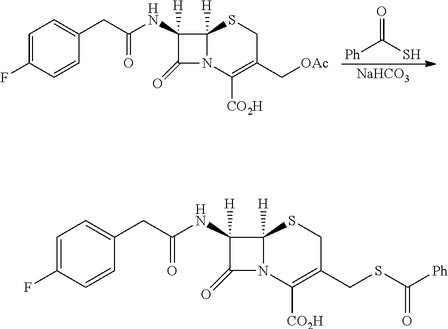

The title compound was isolated as an off-white solid (309 mg, 64%) following the general procedure for substitution described above. ¹H NMR (300 MHz, DMSO-d₆): δ 9.06 (d, J=8.3 Hz, 1H), 7.93-7.90 (m, 2H), 7.69 (br t, J=7.3 Hz, 1H), 7.57-7.52 (m, 2H), 7.30-7.26 (m, 2H), 7.14-7.70 (m, 2H), 5.57 (dd, J=8.3 Hz, J=4.8 Hz, 1H), 5.01 (d, J=4.8 Hz, 1H), 4.27 (d, J=13.1 Hz, 1H), 4.02 (d, J=13.1 Hz, 1H), 3.65 (d, J=17.9 Hz, 1H), 3.53 (d, J=14.0 Hz, 1H), 3.41 (d, J=14.0 Hz, 1H), 3.30 (d, J=17.8 Hz, 1H).

### Example 20

### Sodium (6R,7R)-3-(benzoylthiomethyl)-7-(2-(3-methoxyphenyl)acetamido)-8-oxo-5-thia-1-azabicyclo[4.2.0]oct-2-ene-2-carboxylate

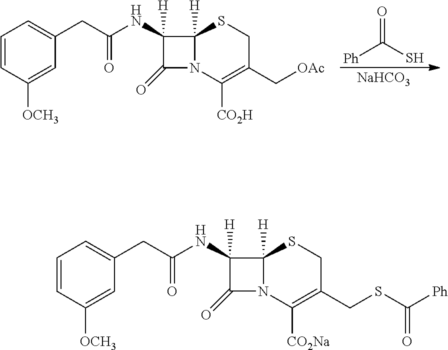

The title compound was isolated as an off-white solid (157.8 mg, 43%) following a modification of the general procedure for substitution described above. The reaction mixture was filtered to isolate the product without prior acidification. ¹H NMR (300 MHz, DMSO-d6): δ 9.03 (d, J=8.4 Hz, 1H), 7.92 (d, J=7.4 Hz, 2H), 7.69 (t, J=7.4 Hz, 1H), 7.55 (t, J=7.4 Hz, 2H), 7.18 (t, J=7.8 Hz, 1H), 6.85-6.73 (m, 3H), 5.57 (dd, J=8.4, 4.8 Hz, 1H), 5.01 (d, J=4.8 Hz, 1H), 4.27 (d, J=13.0 Hz, 1H), 4.05 (d, J=13.0 Hz, 1H), 3.71 (s, 3H), 3.64 (d, J=17.7 Hz, 1H), 3.52 (d, J=13.7 Hz, 1H), 3.43 (d, J=13.7 Hz, 1H), 3.29 (d, J=17.7 Hz, 1H).

### Example 21

### (6R,7R)-7-((Z)-2-(2-aminothiazol-4-yl)-2-(methoxyimino)acetamido)-3-(benzoylthiomethyl)-8-oxo-5-thia-1-azabicyclo[4.2.0]oct-2-ene-2-carboxylic acid

### (6R,7R)-7-((E)-2-(2-aminothiazol-4-yl)-2-(methoxyimino)acetamido)-3-(benzoylthiomethyl)-8-oxo-5-thia-1-azabicyclo[4.2.0]oct-2-ene-2-carboxylic acid

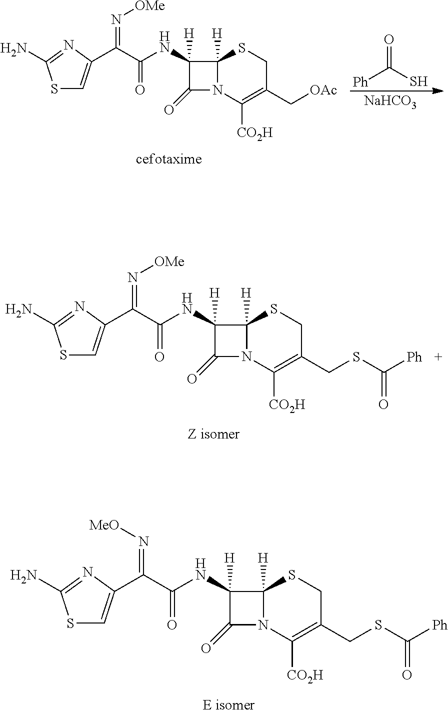

The general procedure for substitution described above afforded the title compound as an off-white solid (113 mg, 22%) which was determined to be a mixture of oxime stereoisomers. ¹H NMR (300 MHz, DMSO-d₆): δ 9.56 (d, J=7.9 Hz, 1H), 9.39 (d, J=8.5 Hz, 1H), 7.93-7.10 (m, 12H), 5.70 (m, 2H), 5.08 (m, 2H), 4.32-4.26 (m, 2H), 4.04-3.28 (m, 16H).

The above-described embodiments are intended to be examples only. Alterations, modifications and variations can be effected to the particular embodiments by those of skill in the art without departing from the scope, which is defined solely by the claims appended hereto.

1. A compound of Formula (I) for use in inhibiting a β-lactamase and/or preventing or treating bacterial resistance to an antibiotic: 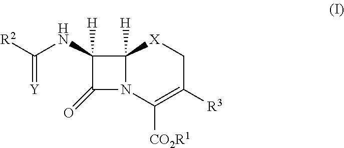
wherein
X is selected from O, S, S═O, SO₂, C═O, C═S, CR⁴R⁵, where R⁴ and R⁵ are independently H or C₁-C₅ alkyl, or NR⁶ where R⁶ is where R⁶ is C(═O)R⁷ or SO₂R⁷ and R⁷ is H or lower alkyl;
Y is selected from O or S;
R¹ is selected from H and a pharmaceutically acceptable cation;
R² is selected from:
—CH₂-aryl, —CH₂dihydro-aryl or —CH₂-heteroaryl,
—(CR′R″)ₙ-aryl or —-(CR′R″)ₙ-heteroaryl, where n=0, 1, 2, 3 or 4 and where R′ and R″ are independently C₁-C₅ alkyl, hydroxy, alkoxy, or cyano,
—C(═N—OR^{′″})-aryl, —C(═N—OR^{′″})-heteroaryl or —C(CH₃)₂—C(═O)OR^{′″} where R^{′″} is H, —C₁-C₅ alkyl, fluoromethyl, CH₂C(CH₃)₂CO₂H or CH₂CO₂H,
cyanomethyl, cyanomethylthiomethyl or dihalomethylthiomethyl, trihalomethylthiomethyl, or
a radical found in a cephalosporin antibiotic, for example, any one of the following 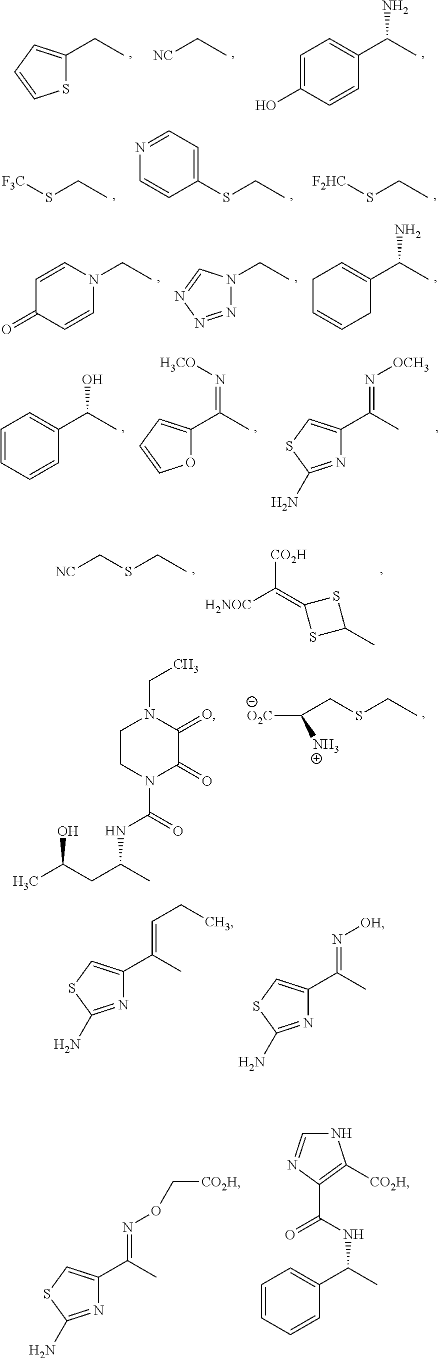 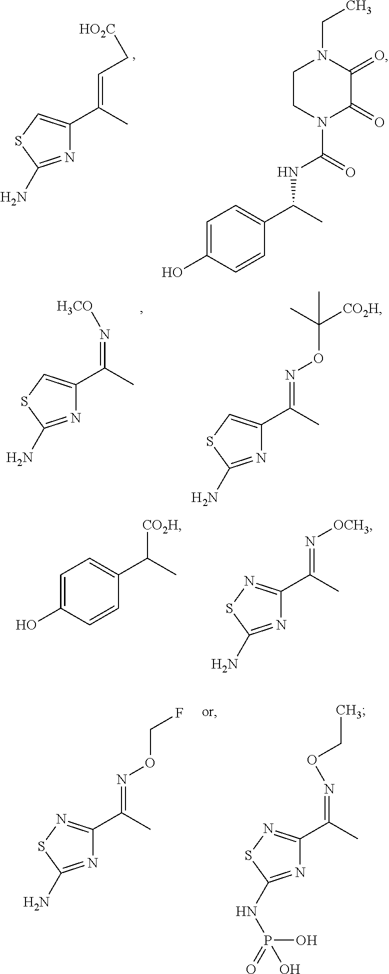
R³ is CR⁸R⁹Z, wherein R⁸ is H or —C₁-C₅ alkyl, R⁹ is H or —C₁-C₅ alkyl, and Z is selected from —S—C(═O)R¹⁰, —S—C(═S)R¹⁰, —SR¹⁰, —SOR¹⁰, —SO₂R¹⁰ or R¹⁰ where R¹⁰ is selected from aryl, preferably having 6 or more carbons, heteroaryl, preferably having 6 or more ring atoms, —(CH₂)ₙ-aryl (where n=1 to 5), —(CH₂)ₙ-heteroaryl (where n=1 to 5), —O(CH₂)ₙH (where n=1 to 5), —S(CH₂)ₙH (where n=1 to 5), —O(CH₂)ₙaryl (where n=0 to 5), —S(CH₂)ₙaryl (where n=0 to 5), —O(CH₂)ₙheteroaryl (where n=0 to 5) or —S(CH₂)ₙheteroaryl (where n=0 to 5), or —O—NHC(═O)R¹¹, —N(SO₂R¹¹)₂ or —OSO₂R¹¹ wherein R^{H} is selected from —C₁-C₅ alkyl, —CF₃, —CCl₃, aryl, heteroaryl, —(CH₂)ₙ-aryl (wherein n=1 to 5), —(CH₂)ₙ-heteroaryl (wherein n=1 to 5), —O(CH₂)ₙH (wherein n=1 to 5), —S(CH₂)ₙH (wherein n=1 to 5), —O(CH₂)ₙaryl (wherein n=0 to 5), —S(CH₂)ₙaryl (wherein n=0 to 5), —O(CH₂)ₙheteroaryl (wherein n=0 to 5) or —S(CH₂)ₙheteroaryl (wherein n=0 to 5),
wherein alkyl, aryl and heteroaryl may be substituted or unsubstituted;
or a pharmaceutically acceptable salt or ester thereof.

2. The compound of claim 1, wherein X is S; Y is O; Z is —S—C(═O)R¹⁰, —S—C(═S)R¹⁰, —SR¹⁰, —SOR¹⁰, —SO₂R¹⁰ or R¹⁰ where R¹⁰ is as defined above; R⁸ and R⁹ are each H, and Z is —SC(═O)R¹⁰ or —SC(═S)R¹⁰; and R¹⁰ is aryl having 6 or more carbons or heteroaryl having 6 or ring atoms.

3-6. (canceled)

7. The compound of claim 2, wherein Z is —SC(═O)R¹⁰.

8. (canceled)

9. The compound of claim 2, wherein the aryl or heteroaryl is independently substituted at 1, 2, 3 or 4 positions by alkyl, preferably lower alkyl, carboxy, carboalkoxy, carboxamido, acyl, aryl, hetroaryl, halo, haloalkyl, haloalkoxy, hydroxy, alkyl, heteroalkyl, aryl, heteroaryl, alkoxy, thioalkoxy, amino, alkylamino, amido, cyano, nitro, oxo, carbonyl, alkoxycarbonyl, thiocarbonyl, acyl, formyl, sulfonyl, mercapto, alkylthio, alkyloxy, alkylamino, cycloalkyl, heterocycloalkyl, aryl, heteroaryl, carboxy, amino, alkylamino, dialkylamino, carbamoyl, aryloxy, heteroaryloxy, arylthio, or heteroarylthio.

10-12. (canceled)

13. The compound of claim 9 wherein R³ is:

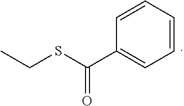

14. The compound of claim 13 wherein R² is substituted or unsubstituted —CH₂-aryl or —CH₂-heteroaryl.

15-22. (canceled)

23. The compound of claim 13, wherein R² is —C(═N—OR^{′″})-aryl or —C(═N—OR^{′″})-heteroaryl, wherein aryl and heteroaryl may be substituted or unsubstituted.

24-29. (canceled)

30. A compound of Formula (I) according to claim 1: 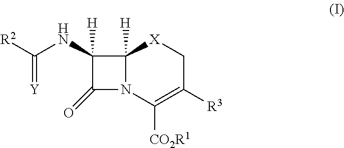
wherein
X is O or S;
Y is O or S;
R¹ is selected from H and a pharmaceutically acceptable cation;
R² is selected from:
—CH₂-aryl, —CH₂dihydro-aryl or —CH₂-heteroaryl,
—(CR′R″)ₙ-aryl or —(CR′R″)ₙ-heteroaryl, where n=0, 1, 2, 3 or 4 and where R′ and R″ are independently C₁-C₅ alkyl, hydroxy, alkoxy, or cyano,
—C(═N—OR^{′″})-aryl, —C(═N—OR^{′″})-heteroaryl or —C(CH₃)₂—C(═O)OR^{′″} where R^{′″} is H, —C₁-C₅ alkyl, fluoromethyl, CH₂C(CH₃)₂CO₂H or CH₂CO₂H,
cyanomethyl, cyanomethylthiomethyl or dihalomethylthiomethyl, trihalomethylthiomethyl, or
a radical found in a cephalosporin antibiotic, for example, any one of the following 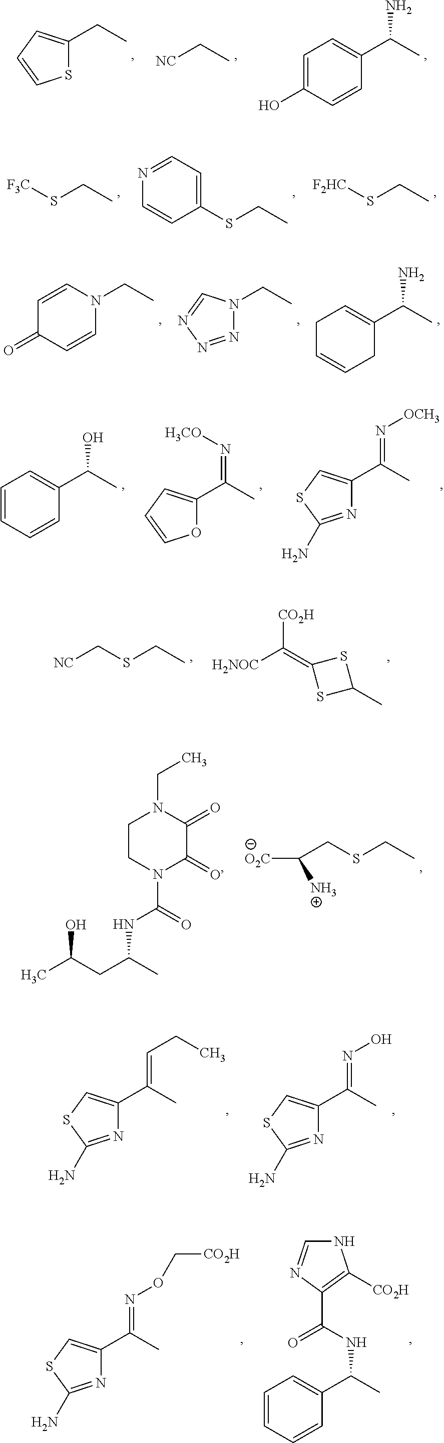 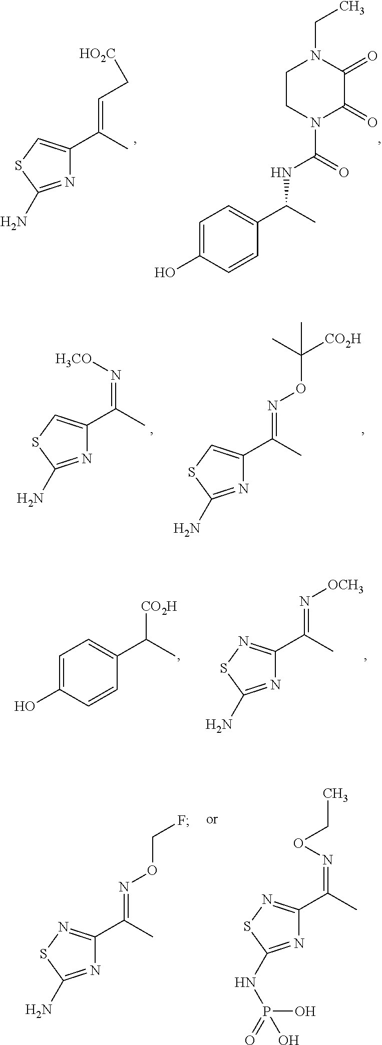
R³ is CR⁸R⁹Z, wherein R⁸ is H or —C₁-C₅ alkyl, R⁹ is H or —C₁-C₅ alkyl, and Z is selected from —S—C(═O)R¹⁰, —S—C(═S)R¹⁰, —SR¹⁰, —SOR¹⁰, —SO₂R¹⁰ or R¹⁰ where R¹⁰ is selected from aryl, preferably having 6 or more carbons, heteroaryl, preferably having 6 or more ring atoms,_(CH₂)ₙ-aryl (where n=1 to 5), (CH₂)ₙ-heteroaryl (where n=1 to 5), O(CH₂)ₙH (where n=1 to 5), S(CH₂)ₙH (where n=1 to 5), O(CH₂)ₙaryl (where n=0 to 5), S(CH₂)ₙaryl (where n=0 to 5), O(CH₂)ₙheteroaryl (where n=0 to 5) or S(CH₂)ₙheteroaryl (where n=0 to 5), or —O—NHC(═O)R¹¹, —N(SO₂R¹¹)₂ or —OSO₂R¹¹ wherein R¹¹ is selected from —C₁-C₅ alkyl, —CF₃, —CCl₃, aryl, heteroaryl, —(CH₂)ₙ-aryl (wherein n=1 to 5), —(CH₂)ₙ-heteroaryl (wherein n=1 to 5), —O(CH₂)ₙH (wherein n=1 to 5), —S(CH₂)ₙH (wherein n=1 to 5), —O(CH₂)ₙaryl (wherein n=0 to 5), —S(CH₂)ₙaryl (wherein n=0 to 5), —O(CH₂)ₙheteroaryl (wherein n=0 to 5) or —S(CH₂)ₙheteroaryl (wherein n=0 to 5),
wherein alkyl, aryl and heteroaryl may be substituted or unsubstituted;
or a pharmaceutically acceptable salt or ester thereof,
with the proviso that the compound is not a known cephalosporin antibiotic as recited herein, such as, ceftiofur, moxalactam, cefaloridin or cefalonium.

31. The compound of claim 30, wherein X is S and Y is O and wherein R¹⁰ is:

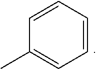

32. (canceled)

33. The compound of claim 31 wherein R² is a radical found in a cephalosporin antibiotic, for example:

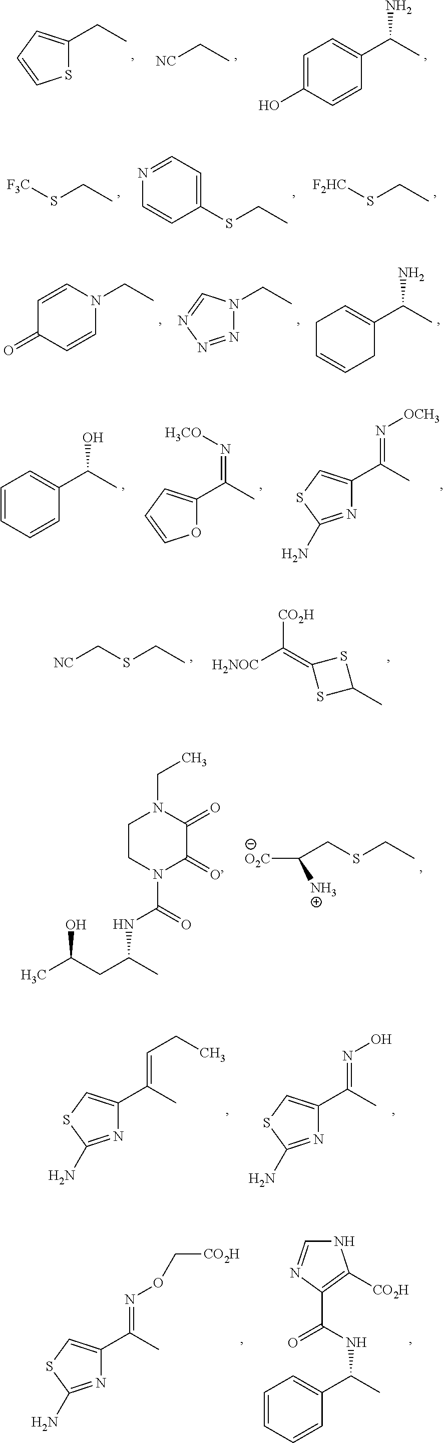

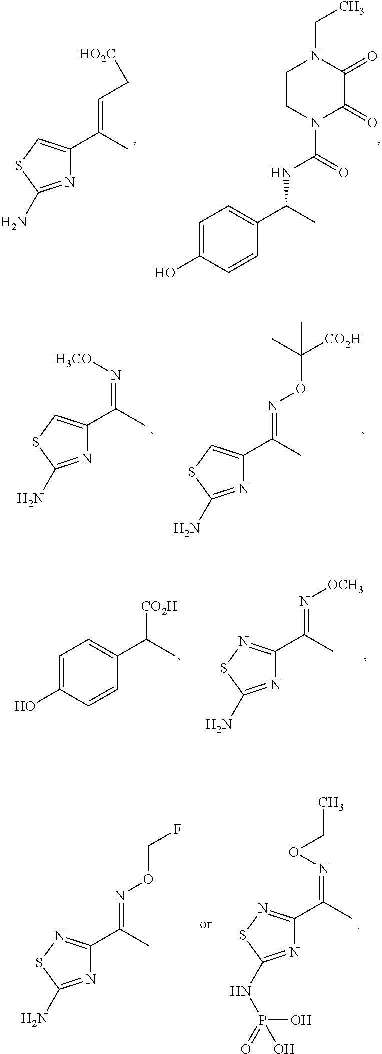

34. The compound of claim 31 wherein R² is selected from:

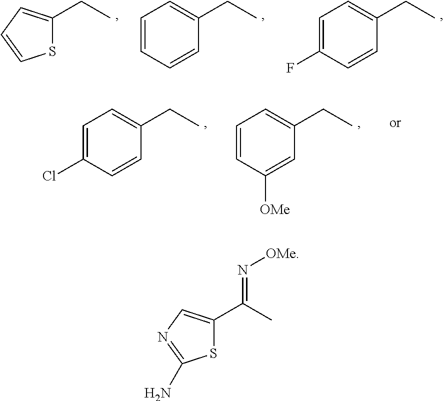

35. A compound according to claim 1 having the structure of Formula (II) 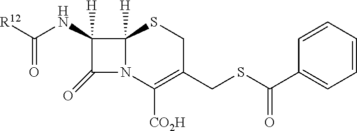
wherein
R¹² is selected from:
—CH₂-aryl, —CH₂dihydro-aryl or —CH₂-heteroaryl,
—(CR′R″)ₙ-aryl or 13 (CR′R″)ₙ-heteroaryl, where n=0, 1, 2, 3 or 4 and where R′ and R″ are independently C₁-C₅ alkyl, hydroxy, alkoxy, or cyano,
—C(═N—OR^{′″})-aryl, —C(═N—OR^{′″})-heteroaryl or —C(CH₃)₂—C(═O)OR^{′″} where R^{′″} is H, —C₁-C₅ alkyl, fluoromethyl, CH₂C(CH₃)₂CO₂H or CH₂CO₂H,
cyanomethyl, cyanomethylthiomethyl or dihalomethylthiomethyl, trihalomethylthiomethyl, or
a radical found in a cephalosporin antibiotic, for example:

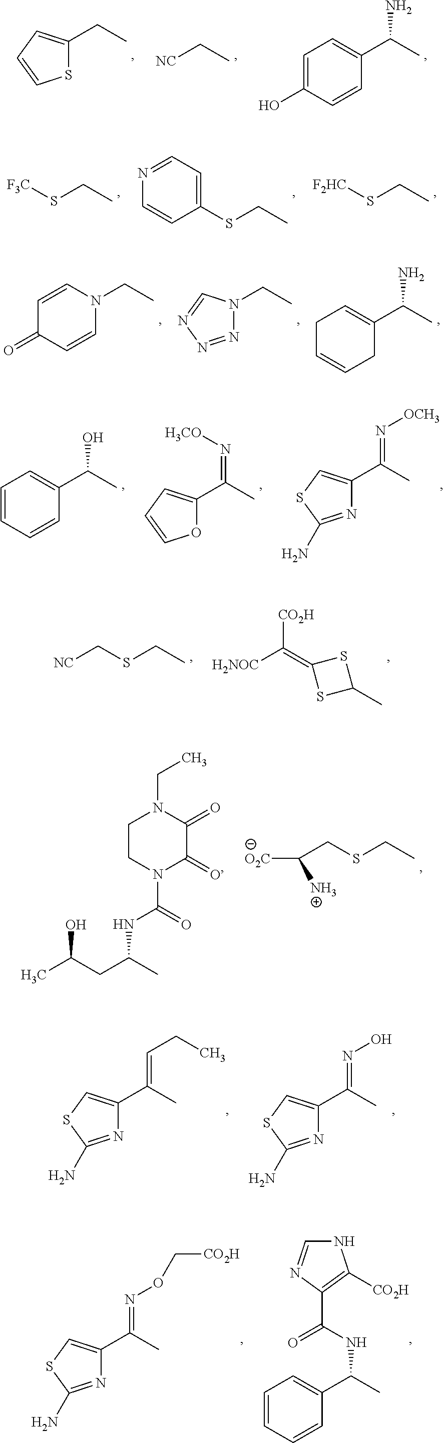

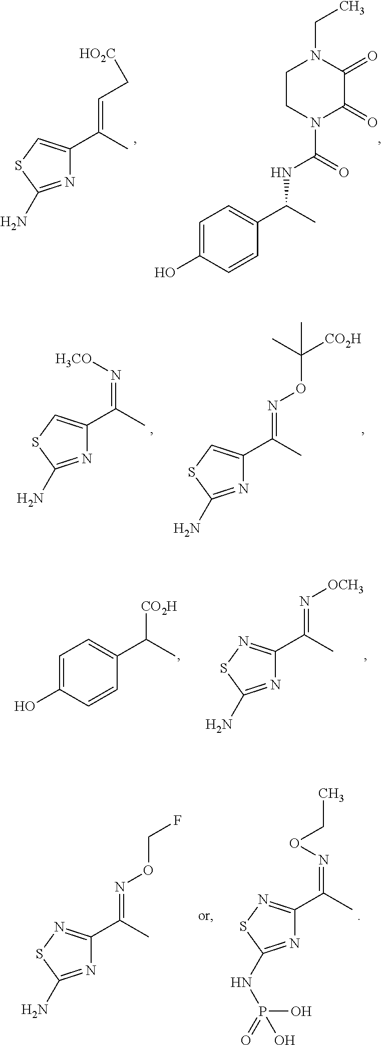

36. The compound of claim 35, wherein R¹² is selected from:

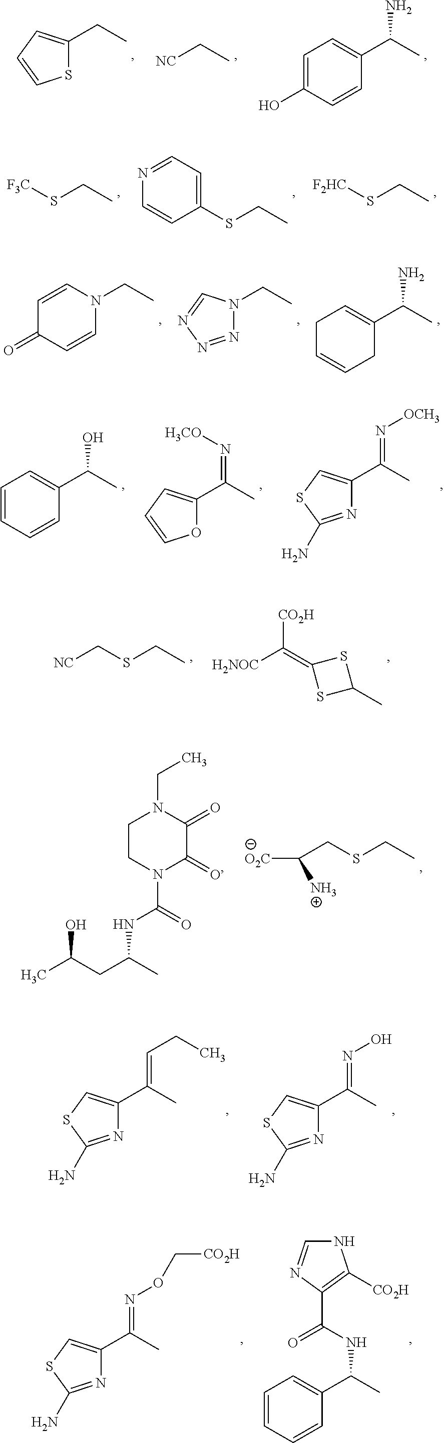

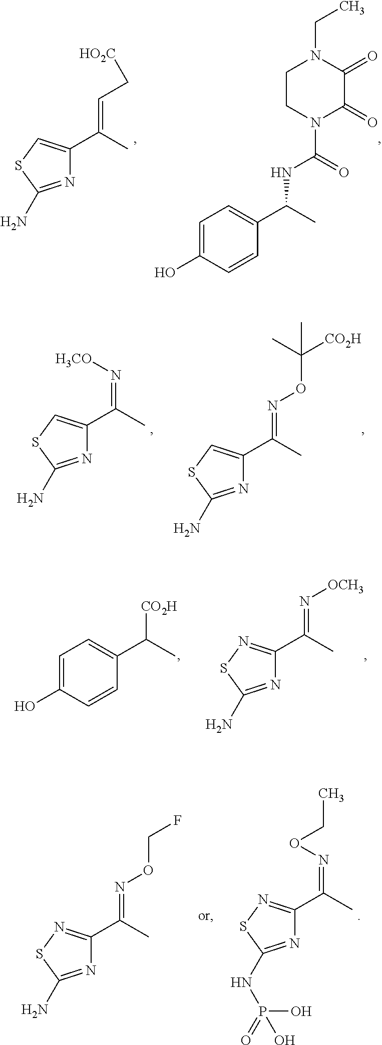

37. The compound of claim 35, wherein R¹² is selected from:

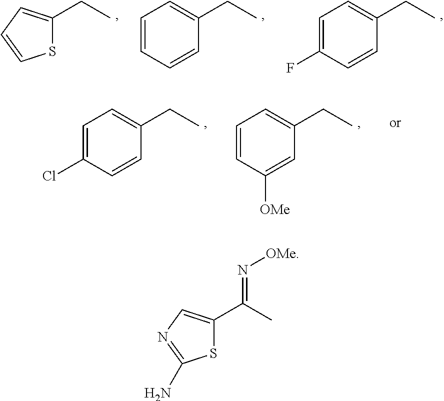

38. A compound according to claim 1 selected from the group consisting of: 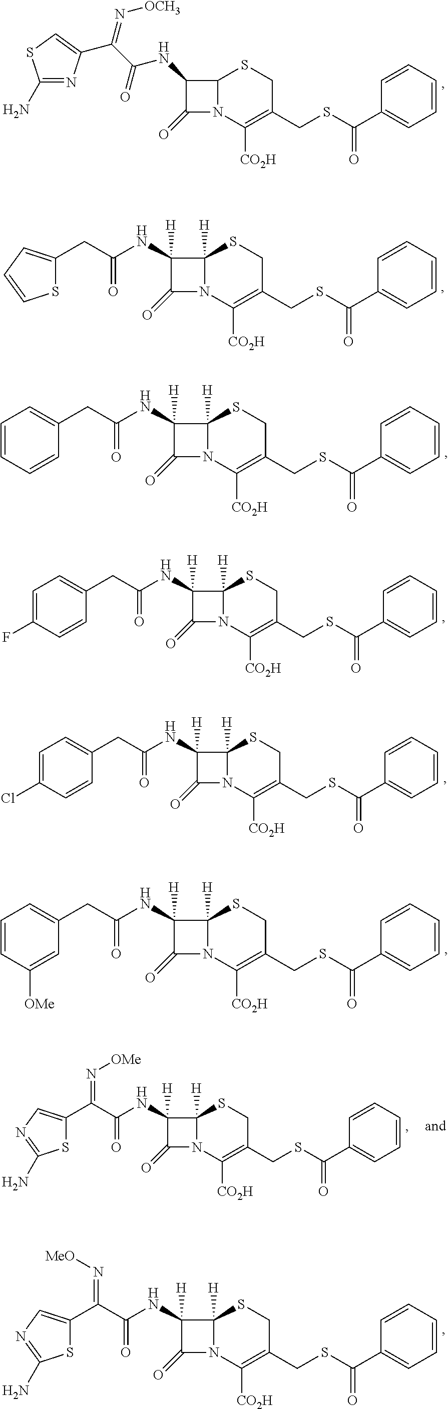
or a pharmaceutically acceptable salt or ester thereof.

39-40. (canceled)

41. A pharmaceutical composition for preventing or treating bacterial resistance to an antibiotic, the composition comprising:
a β-lactamase inhibitory amount of a compound as defined in claim 9, and
a pharmaceutically acceptable excipient.

42. The pharmaceutical composition of claim 41, further comprising a pharmaceutically acceptable β-lactam antibiotic.

43-52. (canceled)

53. A method of treating a bacterial infection and/or preventing or treating bacterial resistance to an antibiotic comprising administering to a patient in need thereof a β-lactamase inhibitory amount of a compound of claim 9 in combination with a therapeutically effective amount of a β-lactam antibiotic.

54. (canceled)

55. The method of claim 53, wherein the β-lactam antibiotic is selected from a penicillin, a cephalosporin, an oxacephem, a carbacephem, a cephamycin, an oxacephamycin, a penem, or a carbapenem.

56-62. (canceled)
